(19) United States
(12) Patent Application Publication
(10) Pub. No.: US 2024/0358781 A1
(43) Pub. Date: Oct. 31, 2024

(54) ALTERED VIRUS

(71) Applicant: Replimune Limited, Oxfordshire (GB)

(72) Inventor: Robert Stuart Coffin, London (GB)

(73) Assignee: Replimune Limited, Oxfordshire (GB)

(21) Appl. No.: 18/769,170

(22) Filed: Jul. 10, 2024

Related U.S. Application Data
(63) Continuation of application No. 16/466,612, filed on Jun. 4, 2019, now Pat. No. 12,059,444.

(30) Foreign Application Priority Data
Jan. 9, 2017 (GB) 1700350.0

(51) Int. Cl. A61K 35/768; A61K 35/763; A61K 45/06; A61P 35/00; C07K 16/28

(52) U.S. Cl. CPC A61K 35/768; A61K 35/763; A61P 35/00; C07K 16/2818; A61K 45/06

(57) ABSTRACT

The present invention relates to an oncolytic virus encoding a CTLA-4 inhibitor, such as an anti-CTLA-4 antibody, or an antigen binding fragment thereof.

### CROSS-REFERENCE TO RELATED APPLICATIONS

This application is a continuation of U.S. patent application Ser. No. 16/466,612 filed Jun. 4, 2019, which is a national phase application under 35 U.S.C. § 371 that claims priority to International Application No. PCT/GB2018/050048 filed Jan. 9, 2018, which claims priority to United Kingdom Patent Application No. 1700350.0, filed Jan. 9, 2017, all of which are incorporated herein by reference in their entirety.

### INCORPORATION OF SEQUENCE LISTING

The instant application contains a Sequence Listing, named “SeqLst.XML” (72,741 bytes; created Jul. 9, 2024) which has been submitted electronically in XML format and is hereby incorporated by reference in its entirety.

### FIELD OF THE INVENTION

The invention relates to an oncolytic immunotherapeutic agent and to the use of the oncolytic immunotherapeutic agent in treating cancer.

### BACKGROUND TO THE INVENTION

Viruses have a unique ability to enter cells at high efficiency. After entry into cells, viral genes are expressed and the virus replicates. This usually results in the death of the infected cell and the release of the antigenic components of the cell as the cell ruptures as it dies. As a result, virus mediated cell death tends to result in an immune response to these cellular components, including both those derived from the host cell and those encoded by or incorporated into the virus itself and enhanced due to the recognition by the host of so called damage associated molecular patterns (DAMPs) which aid in the activation of the immune response.

Viruses also engage with various mediators of the innate immune response as part of the host response to the recognition of a viral infection through e.g. toll-like receptors and cGAS/STING signalling and the recognition of pathogen associated molecular patterns (PAMPs) resulting in the activation of interferon responses and inflammation which are also immunogenic signals to the host. These immune responses may result in the immunogenic benefit to cancer patients such that immune responses to tumor antigens provide a systemic overall benefit resulting in the treatment of tumors which have not been infected with the virus, including micro-metastatic disease, and providing vaccination against relapse.

The combined direct (‘oncolytic’) effects of the virus, and immune responses against tumor antigens (including non-self ‘neo-antigens’, i.e. derived from the particular mutated genes in individual tumors) is termed ‘oncolytic immunotherapy’.

Viruses may also be used as delivery vehicles (‘vectors’) to express heterologous genes inserted into the viral genome in infected cells. These properties make viruses useful for a variety of biotechnology and medical applications. For example, viruses expressing heterologous therapeutic genes may be used for gene therapy. In the context of oncolytic immunotherapy, delivered genes may include those encoding specific tumor antigens, genes intended to induce immune responses or increase the immunogenicity of antigens released following virus replication and cell death, genes intended to shape the immune response which is generated, genes to increase the general immune activation status of the tumor, or genes to increase the direct oncolytic properties (i.e. cytotoxic effects) of the virus. Importantly, viruses have the ability to deliver encoded molecules which are intended to help to initiate, enhance or shape the systemic anti-tumor immune response directly and selectively to tumors, which may have benefits of e.g. reduced toxicity or of focusing beneficial effects on tumors (including those not infected by the virus) rather than off-target effects on normal (i.e. non-cancerous) tissues as compared to the systemic administration of these same molecules or systemic administration of other molecules targeting the same pathways.

It has been demonstrated that a number of viruses including, for example, herpes simplex virus (HSV) have utility in the oncolytic treatment of cancer. HSV for use in the oncolytic treatment of cancer must be disabled such that it is no longer pathogenic, but can still enter into and kill tumor cells. A number of disabling mutations to HSV, including disruption of the genes encoding ICP34.5, ICP6, and/or thymidine kinase, have been identified which do not prevent the virus from replicating in culture or in tumor tissue in vivo, but which prevent significant replication in normal tissue. HSVs in which only the ICP34.5 genes have been disrupted replicate in many tumor cell types in vitro, and replicate selectively in tumor tissue, but not in surrounding tissue, in mouse tumor models. Clinical trials of ICP34.5 deleted, or ICP34.5 and ICP6 deleted, HSV have also shown safety and selective replication in tumor tissue in humans.

As discussed above, an oncolytic virus, including HSV, may also be used to deliver a therapeutic gene in the treatment of cancer. An ICP34.5 deleted virus of this type additionally deleted for ICP47 and encoding a heterologous gene for GM-CSF has also been tested in clinical trials, including a phase 3 trial in melanoma in which safety and efficacy in man was shown. GM-CSF is a pro-inflammatory cytokine which has multiple functions including the stimulation of monocytes to exit the circulation and migrate into tissue where they proliferate and mature into macrophages and dendritic cells. GM-CSF is important for the proliferation and maturation of antigen presenting cells, the activity of which is needed for the activation of an anti-tumor immune response. The trial data demonstrated that tumor responses could be seen in injected tumors, and to a lesser extent in uninjected tumors. Responses tended to be highly durable (months-years), and a survival benefit appeared to be achieved in responding patients. Each of these indicated engagement of the immune system in the treatment of cancer in addition to the direct oncolytic effect. However, this and other data with oncolytic viruses generally showed that not all tumors respond to treatment and not all patients achieve a survival advantage. Thus, improvements to the art of oncolytic therapy are clearly needed.

Recently it has been shown that oncolytic immunotherapy can result in additive or synergistic therapeutic effects in conjunction with immune co-inhibitory pathway blockade (i.e. inhibition or ‘antagonism’ of immune checkpoint pathways, also termed immune co-inhibitory pathways). Immune co-inhibitory pathway blockade is intended to block host immune inhibitory mechanisms which usually serve to prevent the occurrence of auto-immunity. However, in cancer patients these mechanisms can also serve to inhibit the induction of or block the potentially beneficial effects of any immune responses induced to tumors.

Systemic blockade of these pathways by agents targeting cytotoxic T lymphocyte-associated molecule-4 (CTLA-4), PD-1 or PD-L1 have shown efficacy in a number of tumor types, including melanoma and lung cancer. However, unsurprisingly, based on the mechanism of action, off target toxicity can occur due to the induction of auto-immunity. Even so, these agents are sufficiently tolerable to provide considerable clinical utility. Other immune co-inhibitory pathway and related targets for which agents (mainly antibodies) are in development include LAG-3, TIM-3, VISTA, CSF1R, IDO, CEACAM1, CD47. Optimal clinical activity of these agents, for example PD1, PDL1, LAG-3, TIM-3, VISTA, CSF1R, IDO, CD47, CEACAM1, may require systemic administration or presence in all tumors due to the mechanism of action, i.e. including targeting of the interface of immune effector cells with tumors or other immune inhibitory mechanisms in/of tumors. In some cases, more localised presence in e.g. just some tumors or in some lymph nodes may also be optimally effective, for example agents targeting CTLA-4.

An alternative approach to increasing the anti-tumor immune response in cancer patients is to target (activate) immune co-stimulatory pathways, i.e. in contrast to inhibiting immune co-inhibitory pathways. These pathways send activating signals into T cells and other immune cells, usually resulting from the interaction of the relevant ligands on antigen presenting cells (APCs) and the relevant receptors on the surface of T cells and other immune cells. These signals, depending on the ligand/receptor, can result in the increased activation of T cells and/or APCs and/or NK cells and/or B cells, including particular sub-types, increased differentiation and proliferation of T cells and/or APCs and/or NK cells and/or B cells, including particular subtypes, or suppression of the activity of immune inhibitory T cells such as regulatory T cells. Activation of these pathways would therefore be expected to result in enhanced anti-tumor immune responses, but it might also be expected that systemic activation of these pathways, i.e. activation of immune responses generally rather than anti-tumor immune responses specifically or selectively, would result in considerable off target toxicity in non-tumor tissue, the degree of such off target toxicity depending on the particular immune co-stimulatory pathway being targeted. Nevertheless agents (mainly agonistic antibodies, or less frequently the soluble ligand to the receptor in question) targeting immune co-stimulatory pathways, including agents targeting GITR, 4-1-BB, OX40, CD40 or ICOS, and intended for systemic use (i.e. intravenous delivery) are in or have been proposed for clinical development.

For many of these approaches targeting immune co-inhibitory or co-inhibitory pathways to be successful, pre-existing immune responses to tumors are needed, i.e. so that a pre-existing immune response can be potentiated or a block to an anti-tumor immune response can be relieved. The presence of an inflamed tumor micro-environment, which is indicative of such an ongoing response, is also needed. Pre-existing immune responses to tumor neo-antigens appear to be particularly important for the activity of immune co-inhibitory pathway blockade and related drugs. Only some patients may have an ongoing immune response to tumor antigens including neoantigens and/or an inflamed tumor microenvironment, both of which are required for the optimal activity of these drugs. Therefore, oncolytic agents which can induce immune responses to tumor antigens, including neoantigens, and/or which can induce an inflamed tumor microenvironment are attractive for use in combination with immune co-inhibitory pathway blockade and immune potentiating drugs. This likely explains the promising combined anti-tumor effects of oncolytic agents and immune co-inhibitory pathway blockade in mice and humans that have so far been observed.

The above discussion demonstrates that there is still much scope for improving oncolytic agents and cancer therapies utilising oncolytic agents, anti-tumor immune responses and drugs which target immune co-inhibitory or co-stimulatory pathways.

### SUMMARY OF THE INVENTION

The present invention provides oncolytic viruses expressing an inhibitor of CTLA-4. The virus may further comprise other immunomodulatory agents. In particular the virus may comprise GM-CSF and/or at least one molecule targeting an immune co-stimulatory pathway. The CTLA-4 inhibitor acts to block a co-inhibitory pathway, i.e. interferes with the interaction between CTLA-4 and B7. GM-CSF aids in the induction of an inflammatory tumor micro-environment and stimulates the proliferation and maturation of antigen presenting cells, including dendritic cells, aiding the induction of an anti-tumor immune responses. These immune responses may be amplified through activation of an immune co-stimulatory pathway or pathways using an immune co-stimulatory pathway activating molecule or molecules also delivered by the oncolytic virus.

Oncolytic viruses replicate within tumors, causing lysis of tumor cells and release of tumor antigens, combined with local inflammation and activation of innate immune responses, all of which are beneficial for the activation of an anti-tumor immune response and for the activity of inhibitors of the CTLA-4/B7 interaction.

Delivery of molecules that inhibit the CTLA-4/B7 interaction directly into an immune response initiating-tumor, including where it would be expected to traffic to draining lymph nodes, focuses immune potentiation by the inhibitor on the tumor and therefore on tumor antigens present within it, reduces systemic toxicity and blocks regulatory T cell (Treg) activation that would otherwise inhibit T-cell activation at the site of immune response initiation. The use of an oncolytic virus to deliver molecules targeting CTLA-4, and optionally molecules targeting immune co-stimulatory pathways to tumors focuses the amplification of immune effects on anti-tumor immune responses, and reduces the amplification of immune responses to non-tumor antigens. Thus, immune cells in tumors and tumor draining lymph nodes are selectively affected by the molecules expressed by the virus rather than immune cells in general. This results in enhanced efficacy of immune cell stimulation, and can also result in reduced off target toxicity. It is also important for focusing the effects of combined systemic immune co-inhibitory pathway blockade and immune co-stimulatory pathway activation on tumors, i.e. such that the amplified immune responses from which co-inhibitory blocks are released are antitumor immune responses rather than responses to non-tumor antigens.

The invention utilizes the fact that, when delivered by an oncolytic virus, the site of action of CTLA-4 blockade and optionally co-stimulatory pathway activation and of GM-CSF expression is in the tumor and/or tumor draining lymph node, but the results of such activation (an amplified systemic anti-tumor-immune response) are systemic. This targets tumors generally, and not only tumors to which the oncolytic virus has delivered the immunomodulatory molecule or molecules. Oncolytic viruses of the invention therefore provide improved treatment of cancer through the generation of improved tumor focused immune responses. The oncolytic virus of the invention also offers improved anti-tumor immune stimulating effects such that the immune-mediated effects on tumors which are not destroyed by oncolysis, including micro-metastatic disease, are enhanced, resulting in more effective destruction of these tumors, and more effective long term anti-tumor vaccination to prevent future relapse and improve overall survival.

Anti-tumor efficacy is improved when an oncolytic virus of the invention is used as a single agent and also when the virus is used in combination with other anti-cancer modalities, including chemotherapy, treatment with targeted agents, radiation and, in preferred embodiments, immune checkpoint blockade drugs (i.e. antagonists of an immune co-inhibitory pathway, for example antibodies against PD1 or PD-L1) and/or agonists of an immune co-stimulatory pathway.

Accordingly, the present invention provides an oncolytic virus encoding a CTLA-4 inhibitor. The CTLA-4 inhibitor is preferably an anti-CTLA-4 antibody or antibody like molecule, or an antigen binding fragment thereof.

The virus may further comprise: (i) a GM-CSF-encoding gene; and/or (ii) an immune co-stimulatory pathway activating molecule or immune co-stimulatory pathway activating molecule-encoding gene. The virus may encode more than one immune co-stimulatory pathway activating molecule/gene.

The immune co-stimulatory pathway activating molecule is preferably GITRL, 4-1-BBL, OX40L, ICOSL or CD40L or a modified version of any thereof. Examples of modified versions include agonists of a co-stimulatory pathway that are secreted rather than being membrane bound, and/or agonists modified such that multimers of the protein are formed.

The virus may be a modified clinical isolate, such as a modified clinical isolate of a virus, wherein the clinical isolate kills two or more tumor cell lines more rapidly and/or at a lower dose in vitro than one or more reference clinical isolates of the same species of virus.

The virus is preferably a herpes simplex virus (HSV), such as HSV1. The HSV typically does not express functional ICP34.5 and/or functional ICP47 and/or expresses the US11 gene as an immediate early gene.

The invention also provides:
a pharmaceutical composition comprising a virus of the invention and a pharmaceutically acceptable carrier or diluent;
the virus of the invention for use in a method of treating the human or animal body by therapy;
the virus of the invention for use in a method of treating cancer, wherein the method optionally comprises administering a further anti-cancer agent;
a product of manufacture comprising a virus of the invention in a sterile vial, ampoule or syringe;
a method of treating cancer, which comprises administering a therapeutically effective amount of a virus or a pharmaceutical composition of the invention to a patient in need thereof, wherein the method optionally comprises administering a further anti-cancer agent;
use of a virus of the invention in the manufacture of a medicament for use in a method of treating cancer, wherein the method optionally comprises administering a further anti-cancer agent.

### BRIEF DESCRIPTION OF THE FIGURES

FIGS. 1A and 1B depict the structures of the viruses used to construct exemplary viruses of the invention that comprise anti-mouse or anti-human CTLA-4 constructs that are codon optimized secreted scFv molecules linked to human or mouse IgG1 Fc regions. The scFvs contain light and heavy variable chains from 9D9 (the initial mouse antibody initially used to validate CTLA-4; WO2007/123737: mouse version) or from ipilimumab. (WO2014/066532; human version) linked by the 15-mer [G₄S]₃ (GGGGSGGGGSGGGGS). The viruses are modified versions of strain HSV1 RH018A (clinical strain 18). The ICP34.5 and ICP47 genes are inactivated in the viruses. The US11 gene is placed under the control of the ICP47 immediate early gene promoter by deletion of the ICP47 promoter. An expression cassette is inserted into the ICP34.5 gene loci. In virus 17, the expression cassette includes the human GM-CSF gene under the control of a CMV promoter and the GALV gene under the control of a RSV promoter. Virus 16 is the same as virus 17, except that human GM-CSF is included instead of mouse GM-CSF. Viruses 25 and 29 are the same as viruses 16 and 17, respectively, except that they each additionally comprise a GFP gene under the control of a MMLV promoter in the expression cassette. Viruses 27 and 31 are the same as viruses 25 and 29, respectively, except that the GFP gene is replaced with mouse anti-CTLA4 and human anti-CTLA4, respectively.

FIGS. 2A-2D depict the structures of the plasmids used to construct the exemplary viruses of the invention.

FIG. 3 shows the structure of anti-mouse or human CTLA-4 constructs that are codon optimized secreted scFv molecules linked to human or mouse IgG1 Fc regions. The scFvs contain the linked ([G₄S]₃) light and heavy variable chains from 9D9 (the initial mouse antibody initially used to validate CTLA-4; US2011044953: mouse version) or from ipilimumab (US20150283234; human version). The resulting structure of the CTLA-4 inhibitor is also shown.

FIG. 4 is a western blot demonstrating that anti-mouse CTLA-4 is expressed from virus 27. The gel used was a reduced denatured PVDF membrane tris-glycine gel. Anti-CTLA-4 was detected using an alkaline phosphatase-tagged anti-mouse IgG1 antibody. Lane 1: spectra broad range ladder; lane 2 virus 27 neat supernatant; lane 3 virus 27 supernatant diluted 1 in 2; lane 4 virus 27 supernatant diluted 1 in 4; lane 5 virus 27 supernatant diluted 1 in 8; lane 6 virus 27 supernatant diluted 1 in 16; lane 7 virus 27 supernatant diluted 1 in 32; lane 8 negative control virus (neat supernatant). The expected size of anti-CTLA-4 (reduced) is 57 kDa.

FIG. 5 shows the superior tumor control and shrinkage in uninjected tumors of a virus expressing anti-mCTLA-4 (virus 27) compared to an otherwise identical virus that does not express CTLA-4 (virus 16). The dose of virus used was 5×10⁴ pfu (50 ul of 1×10⁶ pfu/ml in each case), given three times over one week. This dose level of virus is subtherapeutic for uninjected tumors for virus 16, which allows the benefits of the delivery of the additional molecule encoded by virus 27 to clearly be seen.

FIG. 6 shows the superior tumor control and shrinkage in both injected and uninjected tumors of a virus expressing anti-mCTLA-4 (virus 27) compared to an otherwise identical virus that does not express CTLA-4 (virus 16). The dose of the virus used was 5×10⁴ pfu over one week into the right tumor of a virus expressing anti-mCTLA-4 (virus 27) compared to an otherwise identical virus that does not express CTLA-4 (virus 16). Each line represents a different mouse.

FIG. 7 shows the effect of combined treatment of bilateral mouse A20 tumors using anti-PD1 and virus 27 expressing mGM-CSF, GALVR and anti-mCTLA-4. The top panel shows the effect of anti-PD1 alone on both injected (right) and uninjected (left) tumors. The middle panel shows the effect of virus 27 alone on both injected (right) and uninjected (left) tumors. The bottom panel shows the superior tumor control and shrinkage achieved when anti-PD1 and virus 27 are both injected into the right tumor. The improved anti-tumor effect of the combined treatment is observed in both injected (right) and uninjected (left) tumors. Each line represents a different mouse.

FIG. 8 shows the superior tumor control and shrinkage effects of virus 31 expressing hGM-CSF, GALVR and anti-human CTLA-4 compared to virus 17 expressing only hGM-CSF and GALVR in mouse MC38 tumors in knock-in mice expressing human CTLA-4. The anti-tumor effects of virus 31 are observed when the virus is administered alone or in combination with anti-PD1. Superior tumor control and shrinkage in injected tumors is obtained with virus 31 which expresses anti-human CTLA-4 compared with an otherwise identical virus that does not express anti-human CTLA-4 (left panel). This effect is further enhanced when treatment with the virus is combined with anti-PD1 treatment. Superior tumor control and shrinkage is also observed in uninjected tumors (right panel) when treatment with either virus is combined with anti-PD1 treatment. This improvement is more marked for the virus 31 that expresses anti CTLA-4 than for virus 17 which does not. Each line represents a different mouse.

### BRIEF DESCRIPTION OF THE SEQUENCE LISTING

SEQ ID NO: 1 is the light chain variable region amino acid sequence of the human CTLA-4 antibody used in the Examples.

SEQ ID NOs: 2 is the complete light chain amino acid sequence comprising the light chain variable region amino acid sequence of the human CTLA-4 antibody used in the Examples.

SEQ ID NO: 3 is the heavy chain variable region amino acid sequence of the human CTLA-4 antibody used in the Examples.

SEQ ID NO: 4 is the heavy chain CH1 amino acid sequence of the human CTLA-4 antibody used in the Examples.

SEQ ID NO: 5 is the heavy chain CH2/3 amino acid sequence of the human CTLA-4 antibody used in the Examples.

SEQ ID NO: 6 is the complete heavy chain amino acid sequence of the human CTLA-4 antibody used in the Examples.

SEQ ID NO: 7 is the amino acid sequence of the signal peptide present in the CTLA-4 antibodies of the Examples.

SEQ ID NO: 8 is the amino acid sequence of the linker present between the light chain variable region and the heavy chain variable region in the CTLA-4 antibodies of the Examples.

SEQ ID NO: 9 is the amino acid sequence of the human scFv CTLA-4 antibody of the Examples.

SEQ ID NO: 10 is the nucleotide sequence of the human scFv CTLA-4 antibody of the Examples.

SEQ ID NO: 11 is the light chain variable region amino acid sequence of the murine CTLA-4 antibody used in the Examples.

SEQ ID NO: 12 is the heavy chain variable region amino acid sequence of the murine CTLA-4 antibody used in the Examples.

SEQ ID NO: 13 is the complete heavy chain amino acid sequence of the murine CTLA-4 antibody used in the Examples.

SEQ ID NO: 14 is the amino acid sequence of the murine scFv CTLA-4 antibody of the Examples.

SEQ ID NO: 15 is the nucleotide sequence of the murine scFv CTLA-4 antibody of the Examples.

SEQ ID NO: 16 is the nucleotide sequence of the murine scFv CTLA-4 antibody of the Examples with inserted restriction sites for cloning purposes located at the N and C terminals, that is present in the exemplary virus. The restriction sites are the first six and last eight nucleotides of the sequence.

SEQ ID NO: 17 is the nucleotide sequence of the human scFv CTLA-4 antibody of the Examples with inserted restriction sites for cloning purposes located at the N and C terminals, that is present in the exemplary virus. The restriction sites are the first six and last eight nucleotides of the sequence.

SEQ ID NO: 18 is the nucleotide sequence of mouse GM-CSF.

SEQ ID NO: 19 is the nucleotide sequence of a codon optimized version of mouse GM-CSF.

SEQ ID NO: 20 is the nucleotide sequence of human GM-CSF.

SEQ ID NO: 21 is the nucleotide sequence of a codon optimized version of human GM-CSF.

SEQ ID NO: 22 is the amino acid sequence of mouse GM-CSF.

SEQ ID NO: 23 is the amino acid sequence of human GM-CSF.

SEQ ID NO: 24 is the nucleotide sequence of GALV-R−.

SEQ ID NO: 25 is the nucleotide sequence of a codon optimized version of GALV-R−.

SEQ ID NO: 26 is the amino acid sequence of GALV-R−.

SEQ ID NO: 27 is the nucleotide sequence of a codon optimized version of a human/mouse hybrid membrane bound version of CD40L.

SEQ ID NO: 28 is the amino acid sequence of a human/mouse hybrid membrane bound version of CD40L.

SEQ ID NO: 29 is the nucleotide sequence of a codon optimized version of a multimeric secreted version of human CD40L.

SEQ ID NO: 30 is the amino acid sequence of a multimeric secreted version of human CD40L.

SEQ ID NO: 31 is the nucleotide sequence of a codon optimized version of a multimeric secreted version of mouse CD40L.

SEQ ID NO: 32 is the amino acid sequence of a multimeric secreted version of mouse CD40L.

SEQ ID NO: 33 is the nucleotide sequence of wild-type human CD40L.

SEQ ID NO: 34 is the amino acid sequence of wild-type human CD40L.

SEQ ID NO: 35 is the nucleotide sequence of wild-type mouse CD40L.

SEQ ID NO: 36 is the amino acid sequence of wild-type mouse CD40L.

SEQ ID NO: 37 is the nucleotide sequence of the CMV promoter.

SEQ ID NO: 38 is the nucleotide sequence of the RSV promoter.

SEQ ID NO: 39 is the nucleotide sequence of BGH polyA.

SEQ ID NO: 40 is the nucleotide sequence of SV40 late polyA.

SEQ ID NO: 41 is the nucleotide sequence of rabbit beta-globulin polyA.

SEQ ID NO: 42 is the nucleotide sequence of GFP.

SEQ ID NO: 43 is the nucleotide sequence of retroviral LTR from MMLV.

SEQ ID NO: 44 is the nucleotide sequence of EF1a promoter.

SEQ ID NO: 45 is the nucleotide sequence of SV40 promoter.

SEQ ID NO: 46 is the nucleotide sequence of HGH polyA.

### DETAILED DESCRIPTION OF THE INVENTION

### Oncolytic Virus

The virus of the invention is oncolytic. An oncolytic virus is a virus that infects and replicates in tumor cells, such that the tumor cells are killed. Therefore, the virus of the invention is replication competent. Preferably, the virus is selectively replication competent in tumors. A virus is selectively replication competent in tumor tissue if it replicates more effectively in tumor tissue than in non-tumor tissue. The ability of a virus to replicate in different tissue types can be determined using standard techniques in the art.

The virus of the invention may be any virus which has these properties, including a herpes virus, pox virus, adenovirus, retrovirus, rhabdovirus, paramyxovirus or reovirus, or any species or strain within these larger groups. Viruses of the invention may be wild type (i.e. unaltered from the parental virus species), or with gene disruptions or gene additions. Which of these is the case will depend on the virus species to be used. Preferably the virus is a species of herpes virus, more preferably a strain of HSV, including strains of HSV1 and HSV2, and is most preferably a strain of HSV1. In particularly preferred embodiments the virus of the invention is based on a clinical isolate of the virus species to be used. The clinical isolate may have been selected on the basis of it having particular advantageous properties for the treatment of cancer.

The virus may be a modified clinical isolate, wherein the clinical isolate kills two or more tumor cell lines more rapidly and/or at a lower dose in vitro than one or more reference clinical isolate of the same species of virus. Typically, the clinical isolate will kill two or more tumor cell lines within 48 hours, preferably within 24 hours, of infection at multiplicities of infection (MOI) of less than or equal to 0.1. Preferably the clinical isolate will kill a broad range of tumor cell lines, such as 2, 3, 4, 5, 6, 7, 8, 9, 10 or, for example, all of the following human tumor cell lines: U87MG (glioma), HT29 (colorectal), LNCaP (prostate), MDA-MB-231 (breast), SK-MEL-28 (melanoma), Fadu (squamous cell carcinoma), MCF7 (breast), A549 (lung), MIAPACA-2 (pancreas), CAPAN-1 (pancreas), HT1080 (fibrosarcoma).

In a preferred embodiment, the virus of the invention is a strain selected from:

strain RH018A having the accession number ECCAC 16121904;

strain RH004A having the accession number ECCAC 16121902;

strain RH031A having the accession number ECCAC 16121907;

strain RH040B having the accession number ECCAC 16121908;

strain RH015A having the accession number ECCAC 16121903;

strain RH021A having the accession number ECCAC 16121905;

strain RH023A having the accession number ECCAC 16121906; and

strain RH047A having the accession number ECCAC 16121909.

More preferably, the virus of the invention is a strain selected from:

strain RH018A having the accession number ECCAC 16121904;

strain RH004A having the accession number ECCAC 16121902;

strain RH031A having the accession number ECCAC 16121907;

strain RH040B having the accession number ECCAC 16121908; and

strain RH015A having the accession number ECCAC 16121903.

Most preferably, the virus of the invention is strain RH018A having the accession number EACC 16121904. Any one of the deposited strains may be modified as defined herein.

An HSV of the invention is capable of replicating selectively in tumors, such as human tumors. Typically, the HSV replicates efficiently in target tumors but does not replicate efficiently in non-tumor tissue. This HSV may comprise one or more mutations in one or more viral genes that inhibit replication in normal tissue but still allow replication in tumors. The mutation may, for example, be a mutation that prevents the expression of functional ICP34.5, ICP6 and/or thymidine kinase by the HSV.

In one preferred embodiment, the ICP34.5-encoding genes are mutated to confer selective oncolytic activity on the HSV. Mutations of the ICP34.5-encoding genes that prevent the expression of functional ICP34.5 are described in Chou et al. (1990) Science 250:1262-1266, Maclean et al. (1991) J. Gen. Virol. 72:631-639 and Liu et al. (2003) Gene Therapy 10:292-303, which are incorporated herein by reference. The ICP6-encoding gene and/or thymidine kinase-encoding gene may also be inactivated, as may other genes provided that such inactivation does not prevent the virus infecting or replicating in tumors.

The HSV may contain a further mutation or mutations which enhance replication of the HSV in tumors. The resulting enhancement of viral replication in tumors not only results in improved direct ‘oncolytic’ tumor cell killing by the virus, but also enhances the level of heterologous (i.e. a gene inserted into the virus, in the case of viruses of the invention genes encoding a CTLA-4 inhibitor, GM-CSF and/or an immune co-stimulatory pathway activating molecule(s)) gene expression and increases the amount of tumor antigen released as tumor cells die, both of which may also improve the immunogenic properties of the therapy for the treatment of cancer. For example, in a preferred embodiment of the invention, deletion of the ICP47-encoding gene in a manner that places the US11 gene under the control of the immediate early promoter that normally controls expression of the ICP47 encoding gene leads to enhanced replication in tumors (see Liu et al., 2003, which is incorporated herein by reference).

Other mutations that place the US11 coding sequence, which is an HSV late gene, under the control of a promoter that is not dependent on viral replication may also be introduced into a virus of the invention. Such mutations allow expression of US11 before HSV replication occurs and enhance viral replication in tumors. In particular, such mutations enhance replication of an HSV lacking functional ICP34.5-encoding genes.

Accordingly, in one embodiment the HSV of the invention comprises a US11 gene operably linked to a promoter, wherein the activity of the promoter is not dependent on viral replication. The promoter may be an immediate early (IE) promoter or a non-HSV promoter which is active in mammalian, preferably human, tumor cells. The promoter may, for example, be a eukaryotic promoter, such as a promoter derived from the genome of a mammal, preferably a human. The promoter may be a ubiquitous promoter (such as a promoter of β-actin or tubulin) or a cell-specific promoter, such as tumor-specific promoter. The promoter may be a viral promoter, such as the Moloney murine leukaemia virus long terminal repeat (MMLV LTR) promoter or the human or mouse cytomegalovirus (CMV) IE promoter. HSV immediate early (IE) promoters are well known in the art. The HSV IE promoter may be the promoter driving expression of ICP0, ICP4, ICP22, ICP27 or ICP47.

The genes referred to above, the functional inactivation of which provides the property of tumor selectivity to the virus, may be rendered functionally inactive by any suitable method, for example by deletion or substitution of all or part of the gene and/or control sequence of the gene or by insertion of one or more nucleic acids into or in place of the gene and/or the control sequence of the gene. For example, homologous recombination methods, which are standard in the art, may be used to generate the virus of the invention. Alternatively bacterial artificial chromosome (BAC)-based approaches may be used.

As used herein, the term “gene” is intended to mean the nucleotide sequence encoding a protein, i.e. the coding sequence of the gene. The various genes referred to above may be rendered non-functional by mutating the gene itself or the control sequences flanking the gene, for example the promoter sequence. Deletions may remove one or more portions of the gene, the entire gene or the entire gene and all or some of the control sequences. For example, deletion of only one nucleotide within the gene may be made, resulting in a frame shift. However, a larger deletion may be made, for example at least about 25%, more preferably at least about 50% of the total coding and/or non-coding sequence. In one preferred embodiment, the gene being rendered functionally inactive is deleted. For example, the entire gene and optionally some of the flanking sequences may be removed from the virus. Where two or more copies of the gene are present in the viral genome both copies of the gene are rendered functionally inactive.

A gene may be inactivated by substituting other sequences, for example by substituting all or part of the endogenous gene with a heterologous gene and optionally a promoter sequence. Where no promoter sequence is substituted, the heterologous gene may be inserted such that it is controlled by the promoter of the gene being rendered non-functional. In an HSV of the invention it is preferred that the ICP34.5 encoding-genes are rendered non-functional by the insertion of a heterologous gene or genes and a promoter sequence or sequences operably linked thereto, and optionally other regulatory elements such as polyadenylation sequences, into each the ICP34.5-encoding gene loci.

A virus of the invention is used to express a CTLA-4 inhibitor, and optionally GM-CSF and/or an immune co-stimulatory pathway activating molecule, in tumors. This is typically achieved by inserting a heterologous gene encoding a CTLA-4 inhibitor, and optionally a heterologous gene encoding GM-CSF and/or a heterologous gene encoding the immune co-stimulatory pathway activating molecule, in the genome of a selectively replication competent virus wherein each gene is under the control of a promoter sequence. As replication of such a virus will occur selectively in tumor tissue, expression of the CTLA-4 inhibitor and, if present, expression of the GM-CSF and/or the immune co-stimulatory activating protein by the virus, is also enhanced in tumor tissue as compared to non-tumor tissue in the body. Enhanced expression occurs where expression is greater in tumors as compared to other tissues of the body. Proteins expressed by the oncolytic virus would also be expected to be present in oncolytic virus-infected tumor draining lymph nodes, including due to trafficking of expressed protein and of virus in and on antigen presenting cells from the tumor. Accordingly, the invention provides benefits of expression of the CTLA-4 inhibitor and any co-expressed GM-CSF and/or immune co-stimulatory pathway activating molecule selectively in tumors and tumor draining lymph nodes combined with the anti-tumor effect provided by oncolytic virus replication.

The virus of the invention comprises a CTLA-4 inhibitor. The CTLA-4 inhibitor is a molecule, typically a peptide or protein that binds to CTLA-4 and reduces or blocks signaling through CTLA-4. By reducing CTLA-4 signalling, the inhibitor reduces or removes the block of immune stimulatory pathways by CTLA-4.

The CTLA-4 inhibitor is preferably an antibody or an antigen binding fragment thereof.

The term “antibody” as referred to herein includes whole antibodies and any antigen binding fragment (i.e., “antigen-binding portion”) or single chains thereof. An antibody refers to a glycoprotein comprising at least two heavy (H) chains and two light (kappa)(L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (Clq) of the classical complement system.

The antibody is typically a monoclonal antibody. The antibody may be a chimeric antibody. The antibody is preferably a humanised antibody and is more preferably a human antibody.

The term “antigen-binding fragment” of an antibody refers to one or more fragments of an antibody that retain the ability to specifically bind to CTLA-4. The antigen-binding fragment also retains the ability to inhibit CTLA-4 and hence to reduce or remove the CTLA-4 blockade of a stimulatory immune response. Examples of suitable fragments include a Fab fragment, a F(ab′)2 fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. In a preferred embodiment, the antibody is an scFv. Examples of suitable scFv molecules are disclosed in, for example, WO2007/123737 and WO2014/066532, which are incorporated herein by reference.

The antibody encoding sequences typically encode an antibody or antibody fragment having a N-terminal signal sequence. The signal sequence may have the amino acid sequence shown in SEQ ID NO: 7. For example, this signal sequence is included in a scFv having the amino acid sequence shown in SEQ ID NO: 9 and encoded by the nucleotide sequence shown in SEQ ID NO: 10, and in a scFv having the amino acid sequence shown in SEQ ID NO: 14 and encoded by the nucleotide sequence shown in SEQ ID NO: 15.

In the antibody or antibody fragment, the light chain and heavy chain sequences may be joined by an amino acid linker. The linker typically comprises from about 15 to about 25 amino acids, such as about 18 or 20 amino acids. Any suitable linker may be used, such as linkers comprising glycine and serine residues, for example the amino acid sequence shown in SEQ ID NO: 8. For example, this linker is included in a scFv having the amino acid sequence shown in SEQ ID NO: 9 and encoded by the nucleotide sequence shown in SEQ ID NO: 10, and in a scFv having the amino acid sequence shown in SEQ ID NO: 14 and encoded by the nucleotide sequence shown in SEQ ID NO: 15. Both are preferred antibody fragments for use in the invention.

Other antibody fragments having similar structures are also preferred. Accordingly the virus of the invention may encode an antibody or fragment comprising, or consisting essentially of, a light chain variable region, a linker a heavy chain variable region, a heavy chain CH1 domain, a heavy chain CH2 domain and a heavy chain CH3 domain. The virus may further encode a signal sequence at the N-terminus of the antibody.

The antibodies or antibody fragments of the invention may preferably comprise an Fc region which is an IgG1, IgG2, IgG3 or IgG4 region, more preferably an IgG1 region. Preferably, the antibody is an scFv antibody in which the scFv is linked to IgG heavy chain CH2 and CH3 domains.

A preferred CTLA-4 antibody or fragment comprises the heavy chain variable region shown in SEQ ID NO:3 and/or the light chain variable region shown in SEQ ID NO: 1 or the heavy chain variable region shown in SEQ ID NO:11 and/or the light chain variable region shown in SEQ ID NO: 12. The antibody may comprise the heavy chain CH1 domain having the amino acid sequence shown in SEQ ID NO: 4 and/or the CH2/CH3 domains shown in SEQ ID NO: 5. The antibody may comprise the light chain amino acid sequence shown in SEQ ID NO: 2. An antibody of the invention may alternatively comprise a variant of one of these heavy or light chain variable regions or CDR sequences. For example, a variant may be a substitution, deletion or addition variant of any of the above amino acid sequences.

A variant antibody may comprise 1, 2, 3, 4, 5, up to 10, up to 20, up to 30 or more amino acid substitutions and/or deletions from the specific sequences and fragments discussed above, whilst maintaining the activity of the antibodies described herein. “Deletion” variants may comprise the deletion of, for example, 1, 2, 3, 4 or 5 individual amino acids or of one or more small groups of amino acids such as 2, 3, 4 or 5 amino acids. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid.

The virus of the invention comprises one or more polynucleotide sequence encoding the CTLA-4 inhibitor. The polynucleotide sequence is under the control of a suitable promoter. The virus may comprise a first polynucleotide sequence encoding an antibody heavy chain variable region and a second polynucleotide encoding an antibody light chain variable region. The first polynucleotide may encode a full length heavy chain and/or the second polynucleotide may encode a full length light chain. The first and second polynucleotide may be under the control of a single promoter, optionally with an IRES, or may be under the control of two separate promoters. The separates promoters may be the same or different.

The first polynucleotide may comprise, consist essentially of or consist of the heavy chain variable region encoding sequence shown in SEQ ID NO: 9 and/or the second polynucleotide may comprise, consist essentially of or consist of the heavy chain variable region encoding sequence shown in SEQ ID NO: 10. The first polynucleotide may comprise, consist essentially of or consist of the heavy chain variable region encoding sequence shown in SEQ ID NO: 19 and/or the second polynucleotide may comprise, consist essentially of or consist of the heavy chain variable region encoding sequence shown in SEQ ID NO: 20.

A first and/or second polynucleotide sequences may be a variant of SEQ ID NO: 9, 10, 19 or 20. For example, a variant may be a substitution, deletion or addition variant of either of these nucleic acid sequences. A variant polynucleotide may comprise 1, 2, 3, 4, 5, up to 10, up to 20, up to 30, up to 40, up to 50, up to 75 or more nucleic acid substitutions and/or deletions from SEQ ID NO: 9, 10, 19 or 20.

Suitable variants may be at least 70% homologous to a polynucleotide of any one of nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p 387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids. The codons may be optimized so as to increase expression levels of the encoded proteins in target cells as compared to if the unaltered sequence is used.

The virus of the invention preferably comprises GM-CSF. The sequence of the gene encoding GM-CSF may be codon optimized so as to increase expression levels of the respective proteins in target cells as compared to if the unaltered sequence is used.

The virus of the invention preferably comprises one or more immune co-stimulatory pathway activating molecules and/or one or more genes encoding an immune co-stimulatory pathway activating molecule. Immune co-stimulatory pathway activating molecules include proteins and nucleic acid molecules (e.g. aptamer sequences). Examples of immune co-stimulatory pathway activating molecules include CD40 ligand, GITR ligand, 4-1-BB ligand, OX40 ligand, ICOS ligand, flt3 ligand, TL1A, CD30 ligand, CD70 and single chain antibodies targeting the respective receptors for these molecules (CD40, GITR, 4-1-BB, OX40, ICOS, flt3, DR3, CD30, CD27).

Activators of immune co-stimulatory pathway include mutant or wild type, soluble, secreted and/or membrane bound ligands, and agonistic antibodies including single chain antibodies. Viruses of the invention preferably encode one or more of CD40L, ICOSL, 4-1-BBL, GITRL or OX40L.

Viruses of the invention may encode one or more immune co-stimulatory pathway activating molecules, preferably 1, 2, 3 or 4 immune co-stimulatory pathway activating molecules, more preferably 1 or 2 immune co-stimulatory pathway activating molecules.

The sequence of the gene encoding the immune co-stimulatory activating molecule may be codon optimized so as to increase expression levels of the respective protein(s) in target cells as compared to if the unaltered sequence is used.

The virus of the invention may comprise one or more further heterologous genes in addition to a CTLA-4 inhibitor, and GM-CSF and/or an immune co-stimulatory pathway activating molecule. In a preferred embodiment, the virus may further comprise a fusogenic protein such as GALVR−.

The fusogenic protein may be any heterologous protein capable of promoting fusion of a cell infected with the virus of the invention to another cell. A fusogenic protein, preferably a wild type or modified viral glycoprotein (i.e. modified to increase its fusogenic properties), is a protein which is capable in inducing the cell to cell fusion (syncitia formation) of cells in which it is expressed. Examples of fusogenic glycoproteins include VSV-G, syncitin-1 (from human endogenous retrovirus-W (HERV-W)) or syncitin-2 (from HERVFRDE1), paramyxovirus SV5-F, measles virus-H, measles virus-F, RSV-F, the glycoprotein from a retrovirus or lentivirus, such as gibbon ape leukemia virus (GALV), murine leukemia virus (MLV), Mason-Pfizer monkey virus (MPMV) and equine infectious anemia virus (EIAV) with the R transmembrane peptide removed (R− versions). In a preferred embodiment the fusogenic protein is from GALV and has the R− peptide removed (GALV-R−).

The virus of the invention may optionally comprise multiple copies of the fusogenic protein-encoding gene, preferably 1 or 2 copies. The virus may comprise two or more different fusogenic proteins, including any of the fusogenic proteins listed above.

The fusogenic protein or proteins optionally expressed by a virus of the invention may be identical to a naturally occurring protein, or may be a modified protein.

The fusogenic protein-encoding gene (fusogenic gene) may have a naturally occurring nucleic acid sequence or a modified sequence. The sequence of the fusogenic gene may, for example, be modified to increase the fusogenic properties of the encoded protein, or to provide codon optimisation and therefore increase the efficiency of expression of the encoded protein.

The invention also provides a virus, such as a pox virus or a HSV, preferably HSV1, which expresses at least three heterologous genes, wherein each of the three heterologous genes is driven by a different promoter selected from the CMV promoter, the RSV promoter, the EF1a promoter, the SV40 promoter and a retroviral LTR promoter. The virus may, for example, express four heterologous genes, wherein each of the four heterologous genes is driven by a different promoter selected from the CMV promoter, the RSV promoter, the EF1a promoter, the SV40 promoter and a retroviral LTR promoter. The retroviral LTR is preferably from MMLV. The heterologous genes may be terminated by polyadenylation sequences. The polyadenylation sequences may be the same or different. Preferably each heterologous gene is terminated by a different polyadenylation sequence, which is preferably selected from the BGH, SV40, HGH and RBG polyadenylation sequences. The invention also provides a virus, such as a pox virus or a HSV, preferably HSV1, which expresses at least three heterologous genes, wherein each of the three heterologous genes is terminated by a different polyadenylation sequence selected from the BGH, SV40, HGH and RBG polyadenylation sequences. The virus may, for example, express four heterologous genes terminated by each of the BGH, SV40, HGH and RBG polyadenylation sequences, respectively.

The at least three heterologous genes may, for example, be selected from a CTLA-4 inhibitor, a gene encoding GM-CSF, a gene encoding an immune co-stimulatory pathway activating molecule and a fusogenic gene. Examples of the three heterologous genes are a CTLA-4 inhibitor, a gene encoding GM-CSF and a gene encoding an immune co-stimulatory pathway activating molecule; a CTLA-4 inhibitor, a gene encoding GM-CSF and a fusogenic gene; and a CTLA-4 inhibitor, a gene encoding an immune co-stimulatory pathway activating molecule and a fusogenic gene. The four heterologous genes may, for example, be a CTLA-4 inhibitor, a gene encoding GM-CSF, a gene encoding an immune co-stimulatory pathway activating molecule and a fusogenic gene. The three or four heterologous genes may comprise, for example, two ore more genes encoding immune co-stimulatory pathway activating molecules and/or two ore more fusogenic genes.

In one embodiment, the promoters controlling expression of the three heterologous genes are the CMV, RSV and MMLV promoters. For example, a preferred virus may comprise a GM-CSF gene under the control of a CMV promoter, a GALV gene under the control of a RSV promoter and a CTLA-4 inhibitor under the control of a MMLV promoter.

In one embodiment, the polyadenylation sequence terminating the at least three heterologous genes are SV40, BGH and RBG polyadenylation sequences. controlling expression of the three heterologous genes are the CMV, RSV and MMLV promoters. For example, a preferred virus may comprise a GM-CSF gene terminated by a BGH polyadenylation sequence, a GALV gene terminated by a SV40 polyadenylation sequence and a CTLA-4 inhibitor terminated by a RGB polyadenylation sequence.

Any combination of the various promoters and polyadenylation sequences may be used with any of the heterologous genes. For example, a preferred virus may comprise a GM-CSF gene under the control of a CMV promoter and terminated by a BGH polyadenylation sequence, a GALV gene under the control of a RSV promoter and terminated by a SV40 polyadenylation sequence, and a CTLA-4 inhibitor under the control of a MMLV promoter terminated by a RGB polyadenylation sequence.

### Production of Virus

Viruses of the invention are constructed using methods well known in the art. For example plasmids (for smaller viruses and single and multiple genome component RNA viruses) or BACs (for larger DNA viruses including herpes viruses) encoding the viral genome to be packaged, including the genes encoding the fusogenic and immune stimulating molecules under appropriate regulatory control, can be constructed by standard molecular biology techniques and transfected into permissive cells from which recombinant viruses can be recovered.

Alternatively, in a preferred embodiment plasmids containing DNA regions flanking the intended site of insertion can be constructed, and then co-transfected into permissive cells with viral genomic DNA such that homologous recombination between the target insertion site flanking regions in the plasmid and the same regions in the parental virus occur. Recombinant viruses can then be selected and purified through the loss or addition of a function inserted or deleted by the plasmid used for modification, e.g. insertion or deletion of a marker gene such as GFP or lacZ from the parental virus at the intended insertion site. In a most preferred embodiment the insertion site is the ICP34.5 locus of HSV, and therefore the plasmid used for manipulation contains HSV sequences flanking this insertion site, between which are an expression cassette encoding GM-CSF and the immune co-stimulatory pathway activating molecule. In this case, the parental virus may contain a cassette encoding GFP in place of ICP34.5 and recombinant virus plaques are selected through the loss of expression of GFP. In a most preferred embodiment the US11 gene of HSV is also expressed as an IE gene. This may be accomplished through deletion of the ICP47-encoding region, or by other means.

The CTLA-4 inhibitor, and optionally the GM-CSF encoding sequences and immune co-stimulatory pathway activating molecule encoding sequences and/or additional protein encoding sequence, such as a sequence encoding a fusogenic protein such as GALVR−, are inserted into the viral genome under appropriate regulatory control. This may be under the regulatory control of natural promoters of the virus species of the invention used, depending on the species and insertion site, or preferably under the control of heterologous promoters. Suitable heterologous promoters include mammalian promoters, such as the IEF2a promoter or the actin promoter. More preferred are strong viral promoters such as the CMV IE promoter, the RSV LTR, the MMLV LTR, other retroviral LTR promoters, or promoters derived from SV40. Preferably each exogenous gene (e.g. encoding the GM-CSF and immune co-stimulatory pathway activating molecule) will be under separate promoter control, but may also be expressed from a single RNA transcript, for example through insertion of an internal ribosome entry sites (IRES) between protein coding sequences. RNA derived from each promoter is typically terminated using a polyadenylation sequence (e.g. mammalian sequences such as the bovine or human growth hormone (BGH) poly A sequence, synthetic polyadenylation sequences, the rabbit betaglobin polyadenylation sequence, or viral sequences such as the SV40 early or late polyadenylation sequence).

Each of the heterologous genes in the virus is typically under the control of a promoter. The promoters controlling expression of the heterologous genes may be the same or different. For example, the anti-CTLA-4, and one or more of the GM-CSF, fusogenic gene and immune co-stimulatory pathway activating molecule-encoding gene may each be under the control of the CMV promoter, the RSV promoter, the EF1a promoter, the SV40 promoter or a retroviral LTR promoter. Alternatively, for example, the anti-CTLA-4 may be under the control of a retroviral LTR promoter such as the MMLV promoter, the GM-CSF gene may be under the control of the CMV promoter and/or the fusogenic gene, such as GALVR− may be under the control of the RSV promoter.

### Pharmaceutical Compositions

The invention provides a pharmaceutical composition comprising the virus and a pharmaceutically acceptable carrier or diluent. Suitable carriers and diluents include isotonic saline solutions, for example phosphate-buffered saline. The composition may further comprise other constituents such as sugars or proteins to improve properties such as stability of the product. Alternatively a lyophilized formulation may be used, which is reconstituted in a pharmaceutically acceptable carrier or diluent before use.

The choice of carrier, if required, is frequently a function of the route of delivery of the composition. Within this invention, compositions may be formulated for any suitable route and means of administration. Pharmaceutically acceptable carriers or diluents are those used in compositions suitable for intra-tumoral administration, intravenous/intraarterial administration, administration into the brain or administration into a body cavity (e.g. bladder, pleural cavity or by intraperitoneal administration). The composition may be administered in any suitable form, preferably as a liquid.

The present invention also provides a product of manufacture comprising a virus of the invention in a sterile vial, ampoule or syringe.

### Medical Uses/Methods of Treatment

The invention provides the virus of the invention for use in the treatment of the human or animal body by therapy, particularly for use in a method of treating cancer. The cancer is typically in a mammal, preferably in a human. The virus kills infected tumour cells by lysis and by causing infected tumor cells to fuse with one another. The virus of the invention also elicits a systemic anti-tumor immune response, augmented through the expression of the CTLA-4 inhibitor, and optionally GM-CSF and the immune co-stimulatory pathway activating molecule, which also kills cancer cells.

The invention also provides a method of treating cancer, the method comprising administering a therapeutically effective amount of the virus of the invention to an individual in need thereof.

The invention additionally provides the use of the virus of the invention in the manufacture of a medicament for treating cancer.

The virus of the invention is particularly useful in treating any solid tumor including any adenocarcinoma, carcinoma, melanoma or sarcoma. For example, the virus of the invention is useful in treating head and neck, prostate, breast, ovarian, lung, liver, endometrial, bladder, gall bladder, pancreas, colon, kidney, stomach/gastric, esophageal, or cervical cancers, mesothelioma, melanoma or other skin cancer, lymphoma, glioma or other cancer of the nervous system, or sarcomas such as soft tissue sarcoma.

The virus of the invention may be used to treat malignant tumors, including tumors that have metastasised from the site of the original tumor. In this embodiment, the virus may be administered to the primary tumor or to one or more secondary tumors.

The virus of the invention may be administered in combination with other therapeutic agents, including chemotherapy, targeted therapy, immunotherapy (including immune checkpoint blockade, i.e. administration of one or more antagonist of an immune co-inhibitory pathway, and/or one or more agonist of an immune co-stimulatory pathway) and/or in combination with radiotherapy and/or in combination with any combination of these. The therapeutic agent is preferably an anti-cancer agent.

The virus of the invention may be administered in combination with a second virus, such as a second oncolytic virus.

For example, the therapeutic agent may comprise an immunogen (including a recombinant or naturally occurring antigen, including such an antigen or combination of antigens delivered as DNA or RNA in which it/they are encoded), to further stimulate an immune response, such as a cellular or humoral immune response, to tumor cells, particularly tumor neoantigens. The therapeutic agent may be an agent intended to increase or potentiate an immune response, such as a cytokine, an agent intended to inhibit an immune checkpoint pathway or stimulate an immune potentiating pathway or an agent which inhibits the activity of regulatory T cells (Tregs) or myeloid derived suppressor cells (MDSCs).

The therapeutic agent may be an agent known for use in an existing cancer therapeutic treatment. The therapeutic agent may be radiotherapy or a chemotherapeutic agent. The therapeutic agent may be selected from cyclophosmamide, alkylating-like agents such as cisplatin or melphalan, plant alkaloids and terpenoids such as vincristine or paclitaxel (Taxol), antimetabolites such as 5-fluorouracil, topoisomerase inhibitors type I or II such as camptothecin or doxorubicin, cytotoxic antibiotics such as actinomycin, anthracyclines such as epirubicin, glucocorticoids such as triamcinolone, inhibitors of protein, DNA and/or RNA synthesis such as methotrexate and dacarbaxine, histone deacetylase (HDAC) inhibitors, or any other chemotherapy agent.

The therapeutic agent may be one, or a combination of: immunotherapeutics or immunomodulators, such as TLR agonists; agents that down-regulate T-regulatory cells such as cyclophosphamide; or agents designed to block immune checkpoints or stimulate immune potentiating pathways, including but not limited to monoclonal antibodies, such as a CTLA-4 inhibitor, a PD-1 inhibitor, a PD-L1 inhibitor, a LAG-3 inhibitor, a TIM-3 inhibitor, a VISTA inhibitor, a CSF1R inhibitor, an IDO inhibitor, a CEACAM1 inhibitor, a GITR agonist, a 4-1-BB agonist, a KIR inhibitor, a SLAMF7 inhibitor, an OX40 agonist, a CD40 agonist, an ICOS agonist or a CD47 inhibitor. In a preferred embodiment, the therapeutic agent is a CTLA-4 inhibitor such as an anti-CTLA-4 antibody, a PD1 inhibitor, such as an anti-PD-1 antibody or a PD-L1 inhibitor such as an anti-PD-L1 antibody. Such inhibitors, agonists and antibodies can be generated and tested by standard methods known in the art.

Immunotherapeutic agents may also include bi-specific antibodies, cell based-therapies based on dendritic cells, NK cells or engineered T cells such CAR-T cells or T cells expressing engineered T cell receptors. Immunotherapeutic agents also include agents that target a specific genetic mutation which occurs in tumors, agents intended to induce immune responses to specific tumor antigens or combinations of tumor antigens, including neoantigens and/or agents intended to activate the STING/cGAS pathway, TLR or other innate immune response and/or inflammatory pathway, including intra-tumoral agents.

For example, a virus of the invention may be used: in combination with dacarbazine, a BRAF inhibitor and/or PD1 or PD-L1 blockade to treat melanoma; in combination with taxol, doxorubicin, vinorelbine, cyclophosphamide and/or gemcitabine to treat breast cancer; in combination with 5-fluorouracil and optionally leucovorin, irinoteacan and/or oxaliplatin to treat colorectal cancer; in combination with taxol, carboplatin, vinorelbine and/or gemcitabine, PD-1 or PD-L1 blockade to treat lung cancer; in combination with cisplatin and/or radiotherapy to treat head and neck cancer.

The therapeutic agent may be an inhibitor of the idoleamine 2,3-dioxygenase (IDO) pathway. Examples of IDO inhibitors include epacadostat (INCB024360), 1-methyl-tryptophan, indoximod (1-methyly-D-tryptophan), GDC-0919 or F001287.

The mechanism of action of IDO in suppressing anti-tumor immune responses may also suppress immune responses generated following oncolytic virus therapy. IDO expression is induced by toll like receptor (TLR) activation and interferon-γ both of which may result from oncolytic virus infection. One embodiment of the use of oncolytic virus therapy for cancer treatment includes combination of an oncolytic virus, including a virus expressing a CTLA-4 inhibitor, and optionally GM-CSF and/or an immune co-stimulatory pathway activating molecule or molecules and/or one or more additional protein encoding sequences, such as a sequence encoding a fusogenic protein such as GALVR−, with an inhibitor of the IDO pathway and optionally a further antagonist of an immune co-inhibitory pathway and/or one or more agonist of an immune co-stimulatory pathway, including those targeting PD-1 and/or PD-L1.

Where a therapeutic agent and/or radiotherapy is used in conjunction with a virus of the invention, administration of the virus and the therapeutic agent and/or radiotherapy may be contemporaneous or separated by time. The composition of the invention may be administered before, together with or after the therapeutic agent or radiotherapy. The method of treating cancer may comprise multiple administrations of the virus of the invention and/or of the therapeutic agent and/or radiotherapy. In preferred embodiments, in the case of combination with immune checkpoint blockade or other immune potentiating agents, the virus of the invention is administered once or multiple times prior to the concurrent administration of the immune checkpoint blockade or other immune potentiating agent or agents thereafter, or concurrent with the administration of the immune checkpoint blockade or other immune potentiating agent or agents without prior administration of the virus of the invention.

The virus of the invention may be administered to a subject by any suitable route. Typically, a virus of the invention is administered by direct intra-tumoral injection. Intra-tumoral injection includes direct injection into superficial skin, subcutaneous or nodal tumors, and imaging guided (including CT, MRI or ultrasound) injection into deeper or harder to localize deposits including in visceral organs and elsewhere. The virus may be administered into a body cavity, for example into the pleural cavity, bladder or by intra-peritoneal administration. The virus may be injected into a blood vessel, preferably a blood vessel supplying a tumor.

Therapeutic agents which may be combined with a virus of the invention can be administered to a human or animal subject in vivo using a variety of known routes and techniques. For example, the composition may be provided as an injectable solution, suspension or emulsion and administered via parenteral, subcutaneous, oral, epidermal, intradermal, intramuscular, interarterial, intraperitoneal, intravenous injection using a conventional needle and syringe, or using a liquid jet injection system. The composition may be administered topically to skin or mucosal tissue, such as nasally, intratrachealy, intestinally, sublingually, rectally or vaginally, or provided as a finely divided spray suitable for respiratory or pulmonary administration. In preferred embodiments, the compositions are administered by intravenous infusion, orally, or directly into a tumor.

The virus and/or therapeutic agent may be administered to a subject in an amount that is compatible with the dosage composition that will be therapeutically effective. The administration of the virus of the invention is for a “therapeutic” purpose. As used herein, the term “therapeutic” or “treatment” includes any one or more of the following as its objective: the prevention of any metastasis or further metastasis occurring; the reduction or elimination of symptoms; the reduction or complete elimination of a tumor or cancer, an increase in the time to progression of the patient's cancer; an increase in time to relapse following treatment; or an increase in survival time.

Therapeutic treatment may be given to Stage I, II, III, or IV cancers, preferably Stage II, III or IV, more preferably Stage III or IV, pre- or post-surgical intervention (i.e. following recurrence or incomplete removal of tumors following surgery), preferably before any surgical intervention (either for resection of primary or recurrent/metastatic disease), or following recurrence following surgery or following incomplete surgical removal of disease, i.e. while residual tumor remains.

Therapeutic treatment may be carried out following direct injection of the virus composition into target tissue which may be the tumor, into a body cavity, or a blood vessel. As a guide, the amount of virus administered is in the case of HSV in the range of from 10⁴ to 10¹⁰ pfu, preferably from 10⁵ to 109 pfu. In the case of HSV, an initial lower dose (e.g. 10⁴ to 107 pfu) may be given to patients to seroconvert patients who are seronegative for HSV and boost immunity in those who are seropositive, followed by a higher dose then being given thereafter (e.g. 10⁶ to 109 pfu). Typically up to 20 ml of a pharmaceutical composition consisting essentially of the virus and a pharmaceutically acceptable suitable carrier or diluent may be used for direct injection into tumors, or up to 50 ml for administration into a body cavity (which may be subject to further dilution into an appropriate diluent before administration) or into the bloodstream. However for some oncolytic therapy applications larger or smaller volumes may also be used, depending on the tumor and the administration route and site.

The routes of administration and dosages described are intended only as a guide since a skilled practitioner will be able to determine readily the optimum route of administration and dosage. The dosage may be determined according to various parameters, especially according to the location of the tumor, the size of the tumor, the age, weight and condition of the patient to be treated and the route of administration. Preferably the virus is administered by direct injection into the tumor or into a body cavity. The virus may also be administered by injection into a blood vessel. The optimum route of administration will depend on the location and size of the tumor. Multiple doses may be required to achieve an immunological or clinical effect, which, if required, will be typically administered between 2 days to 12 weeks apart, preferably 3-days to 3 weeks apart. Repeat doses up to 5 years or more may be given, preferably for up to one month to two years dependent on the speed of response of the tumor type being treated and the response of a particular patient, and any combination therapy which may also be being given.

The following Examples illustrate the invention.

### Example 1. Construction of a Virus of the Invention

The virus species used to exemplify the invention is HSV, specifically HSV1.

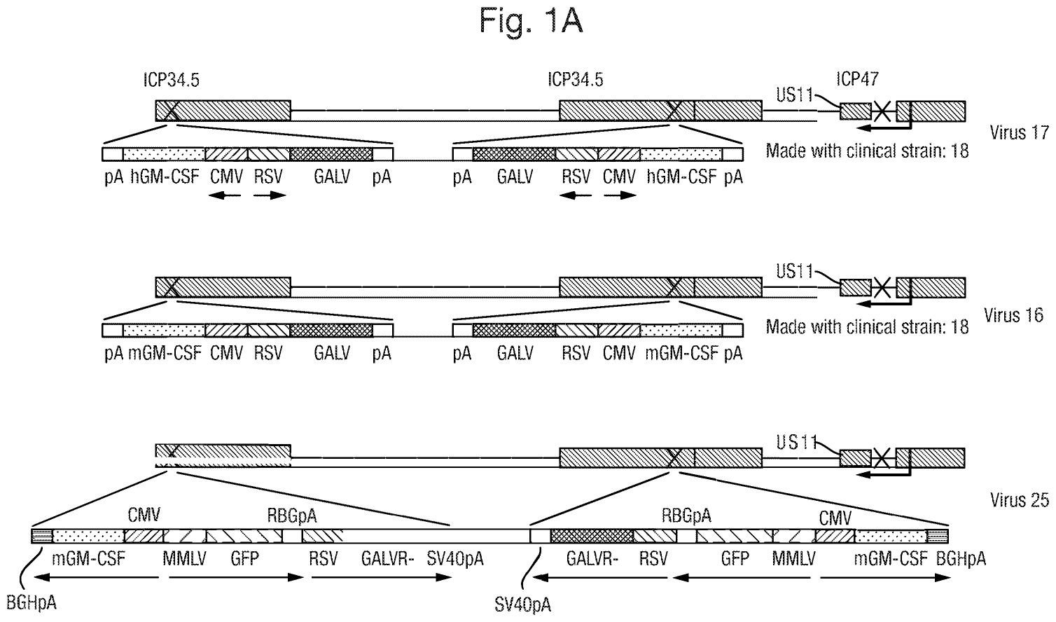

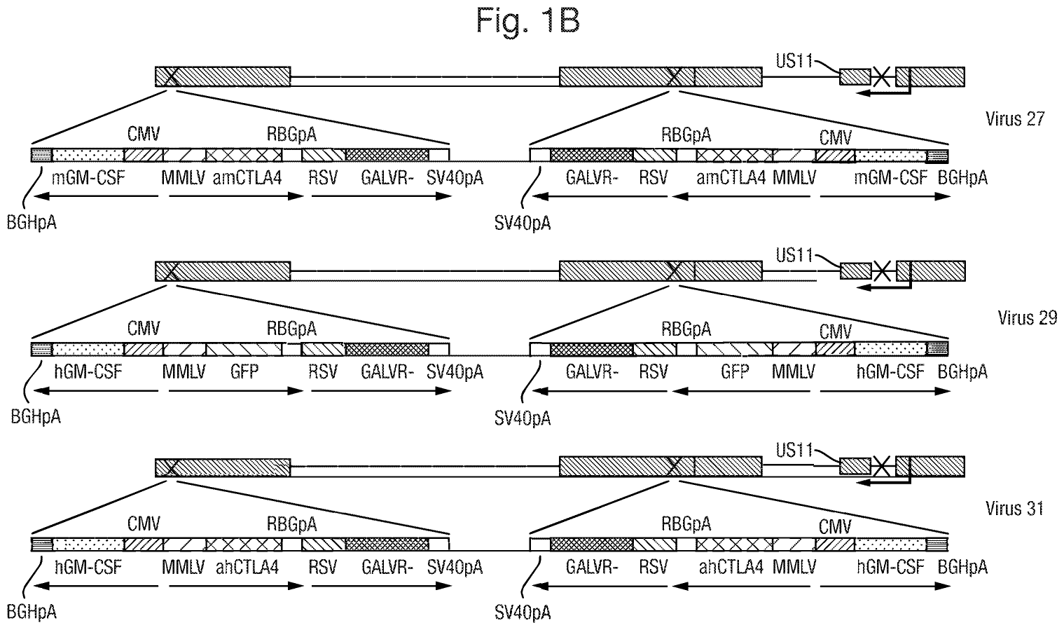

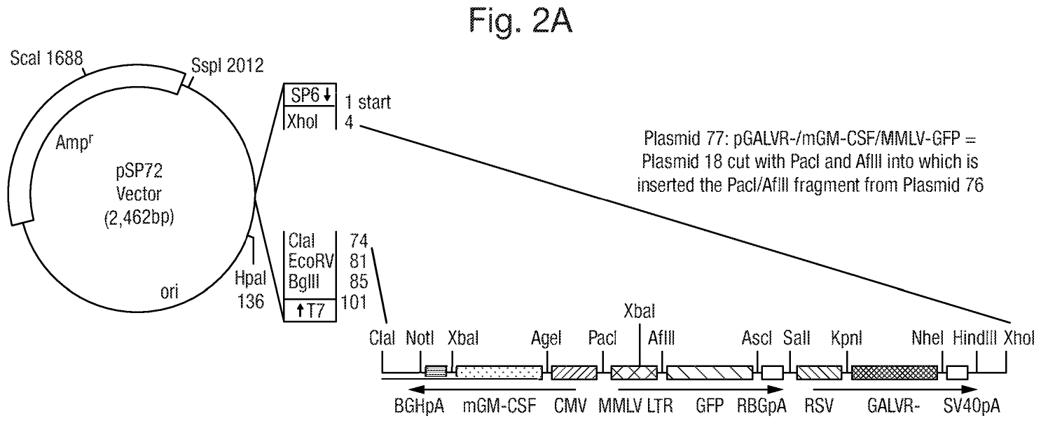

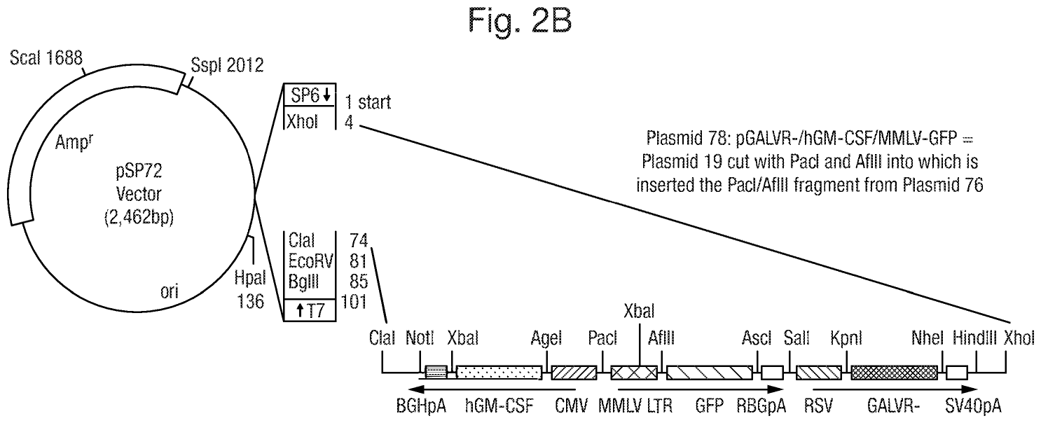

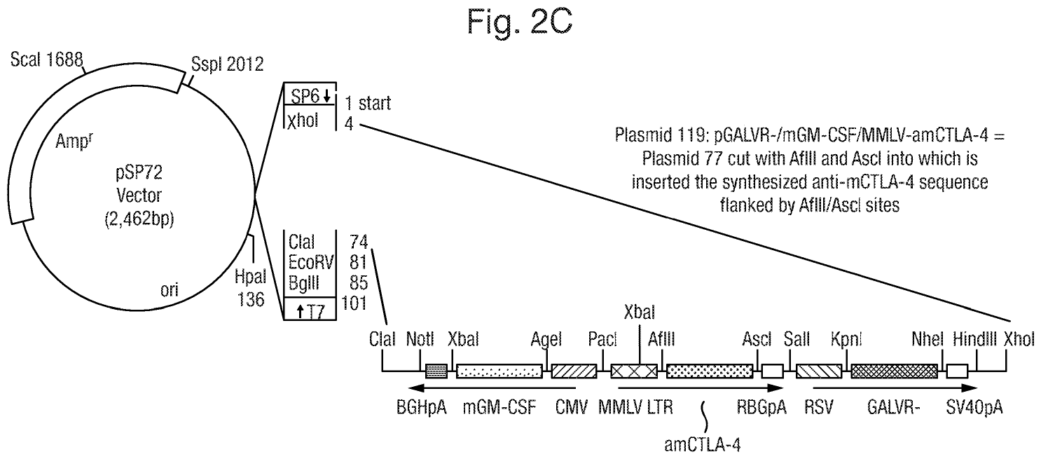

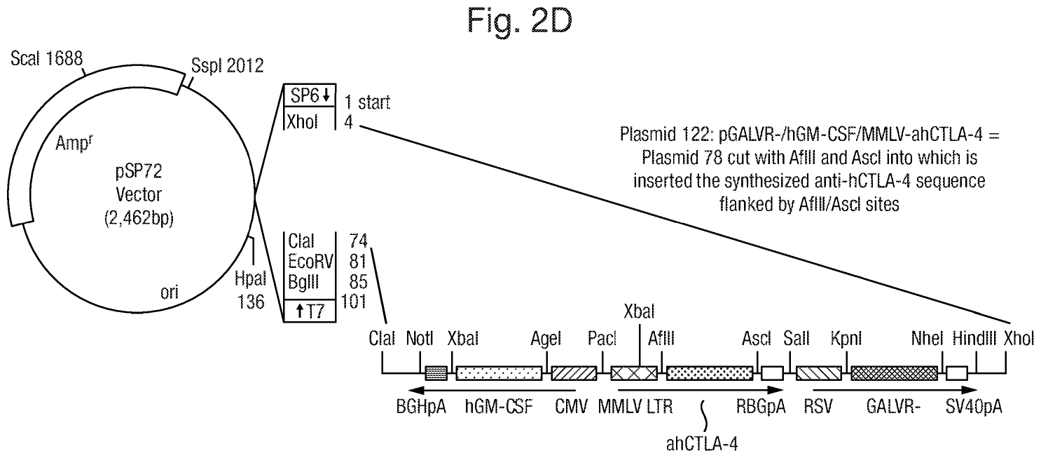

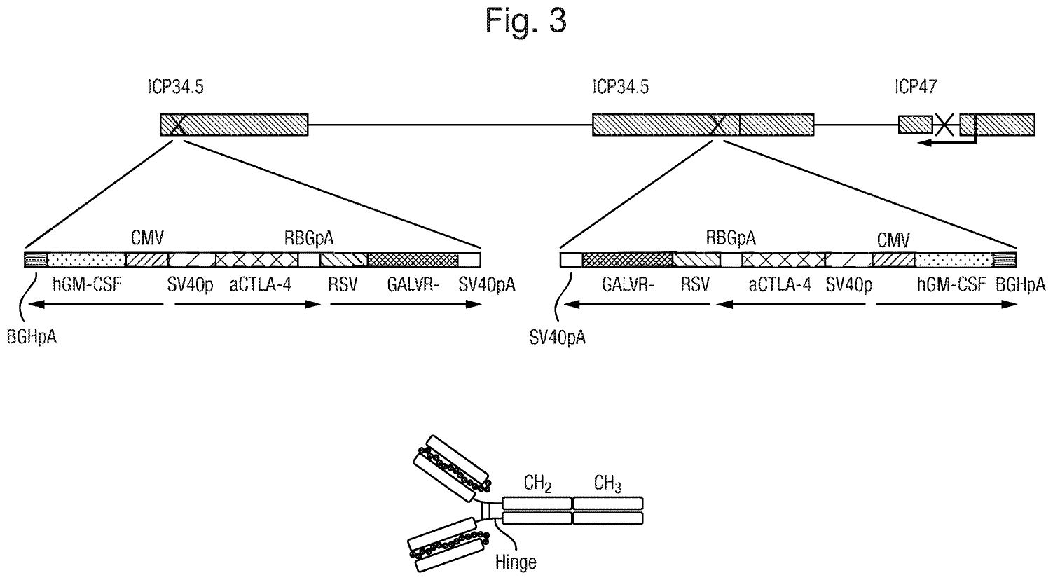

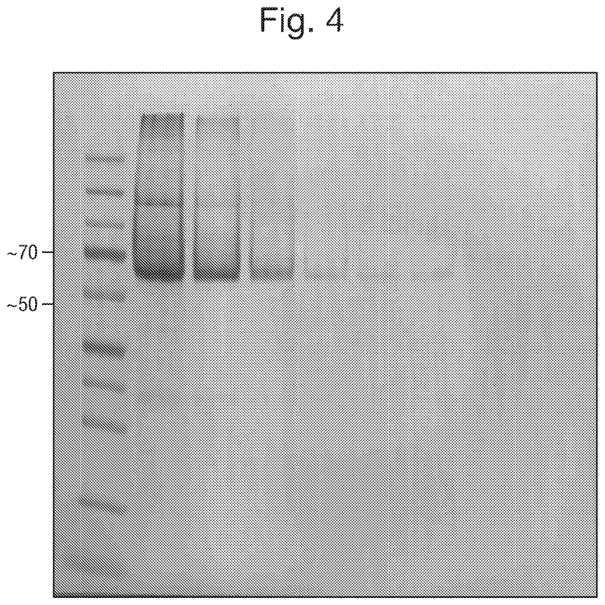

Diagrams of the plasmids used are shown in FIGS. 2A-2D. Diagrams of the viruses are shown in FIGS. 1A and 1B. All viruses were constructed using HSV1 Strain RH018A. The plasmids used for virus construction were generated by a combination of gene synthesis and subcloning, conducted by Genscript Inc.

Viruses expressing anti-mouse CTLA4 together with mouse GM-CSF and GALV were constructed by co-transfection of Plasmid 77 with Virus 16 DNA, so as to insert GFP into Virus 16 by selection of plaques expressing GFP to give Virus 25. GFP was then knocked out of Virus 25 by co-transfection of Virus 25 DNA with Plasmid 119. This gave Virus 27.

Viruses expressing anti-human CTLA4 together with human GM-CSF and GALV were constructed by co-transfection of Plasmid 78 with Virus 17 DNA, so as to insert GFP into Virus 17 by selection of plaques expressing GFP to give Virus 29. GFP was then knocked out of Virus 29 by co-transfection of Virus 29 DNA with Plasmid 122. This gave Virus 31.

Viruses expressing anti-mouse CTLA-4 and co-stimulatory ligands together with mouse GM-CSF and GALV were constructed by co-transfection of a plasmid encoding GFP driven by an SV40 promoter between the mouse GM-CSF and anti-mouse CTLA-4 encoding sequences with Virus 27. GFP was then knocked out of the resulting virus with a plasmid encoding each of the individual mouse co-stimulatory ligands in place of GFP.

Viruses expressing anti-human CTLA-4 and co-stimulatory ligands together with human GM-CSF and GALV were constructed by co-transfection of a plasmid encoding GFP driven by an SV40 promoter between the human GM-CSF and anti-human CTLA-4 encoding sequences with Virus 31. GFP was then knocked out of the resulting virus with a plasmid encoding each of the individual human co-stimulatory ligands in place of GFP.

FIG. 4 shows a western blot demonstrating expression of anti-mouse CTLA-4 from Virus 27.

### Example 2. The Effect of Combined Expression of GALV, GM-CSF and Anti-CTLA4 from an Oncolytic Virus

The utility of the invention is demonstrated in the following way. A20 cells are administered into both flanks of Balb/c mice and the A20 tumors are allowed to grow to approximately 0.5 cm in diameter.

The following treatments are then administered to groups of mice, into one flank of each mouse only (right tumor) 3 times per week for one week:
50 μl of vehicle (1 group);
50 μl of 10⁶ pfu/ml of the HSV with only mouse GM-CSF and GALVR-inserted (Virus 16);
50 μl of 10⁶ pfu/ml of the HSV with GALVR−, mouse GM-CSF and the anti-mouse CTLA-4 antibody inserted (Virus 27);

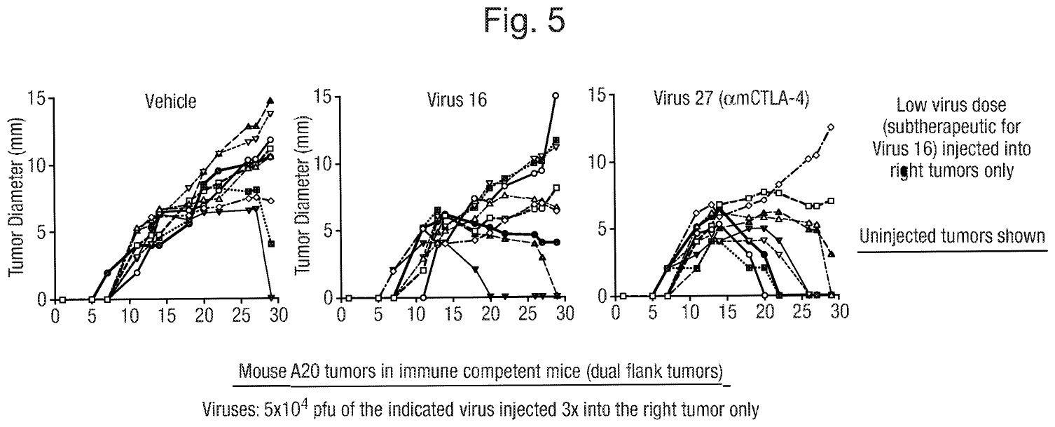

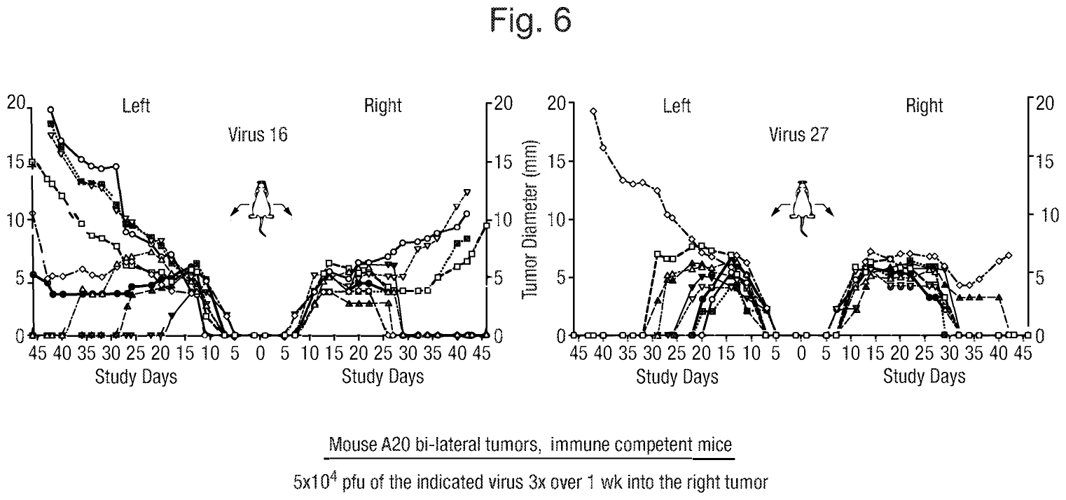

Effects on tumor growth are then observed for up to one month. The dose of virus used was 5×10⁴ pfu (50 ul of 1×10⁶ pfu/ml in each case), given three times over one week. This dose level of virus is subtherapeutic for uninjected tumors for virus 16, which allows the benefits of the delivery of the additional molecules encoded by virus 27 to clearly be seen. FIGS. 5 and 6 show the superior tumor control and shrinkage in uninjected tumors with the virus expressing anti-CTLA-4 compared to with virus 16, which does not express CTLA-4.

### Example 3. The Effect of Combined Expression of GALV, GM-CSF and Anti-CTLA4 from an Oncolytic Virus with Anti-PD-1

A20 cells are administered into both flanks of Balb/c mice and the A20 tumors are allowed to grow to approximately 0.5 cm in diameter.

The following treatments are then administered to groups of mice (10 per group), into one flank of each mouse only 3 times per week for one week:
50 μl of vehicle;
Intraperitoneal anti-mouse PD1 (Bioxcell RMP-1-14 10 mg/kg every three days);
50 μl of 107 pfu/ml of the HSV with GALVR−, mouse GM-CSF and the anti-mouse CTLA-4 antibody inserted (Virus 27)
50 μl of 107 pfu/ml, of the HSV with GALVR−, mouse GM-CSF and the anti-mouse CTLA-4 antibody inserted (Virus 27) together with intraperitoneal anti-mouse PD1 (10 mg/kg every three days) (3 groups).

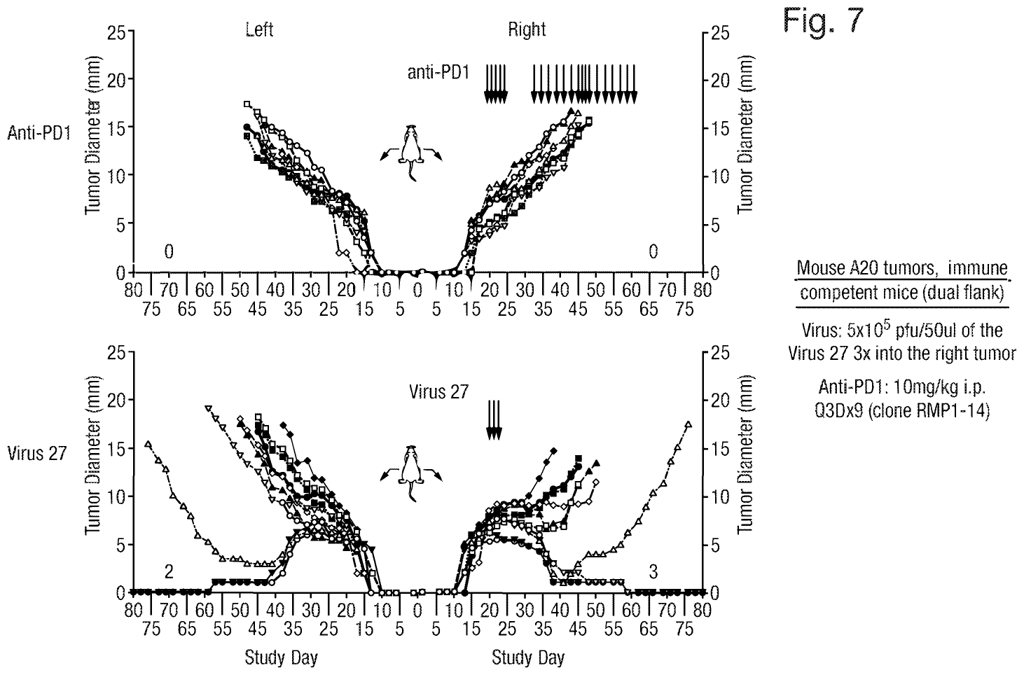

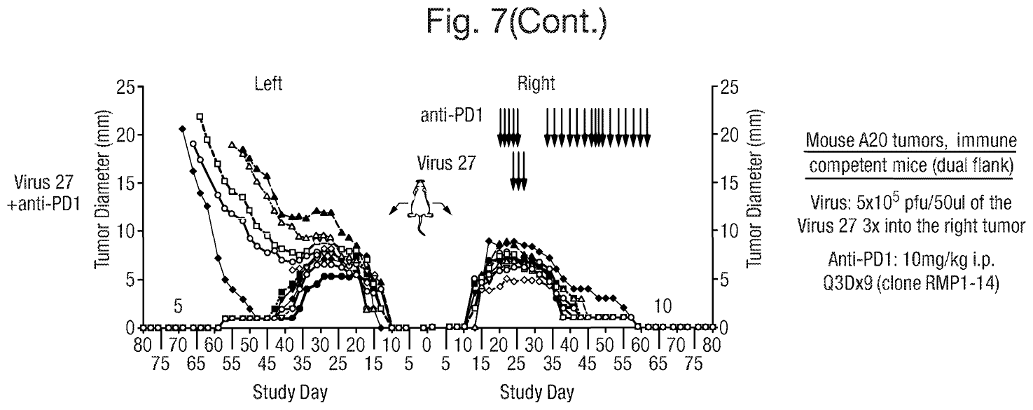

Effects on tumor growth are then observed for up to 80 days. Superior tumor control and shrinkage in both injected and un-injected tumors when treatment with the virus is combined treatment with anti-PD1. This data is shown in FIG. 7.

### Example 4. The Effect of Combined Expression of GALV, GM-CSF and Anti-Human CTLA4 from an Oncolytic Virus Alone and in Combination with Anti-PD-1

MC38 cells are administered into both flanks of C57BL/6 mice engineered by gene editing to express human rather than mouse CTLA-4. This renders the mice susceptible to anti-human CTLA-4 antibodies such as ipilimumab. The MC38 tumors are allowed to grow to approximately 0.5 cm in diameter.

The following treatments are then administered to groups of mice (10 per group), into one flank of each mouse only 3 times per week for two weeks:
50 μl of vehicle;
50 μl of 10⁸ pfu/ml of Virus 17 (i.e. expressing hGM-CSF and GALV);
50 μl of 10⁸ pfu/ml of Virus 31 (i.e. expressing hGM-CSF, GALV and anti-human CTLA-4);
50 μl of 10⁸ pfu/ml of Virus 17 together with intraperitoneal anti-mouse PD1 (10 mg/kg every three days);
50 μl of 10⁸ pfu/ml of Virus 31 together with intraperitoneal anti-mouse PD1 (10 mg/kg every three days).

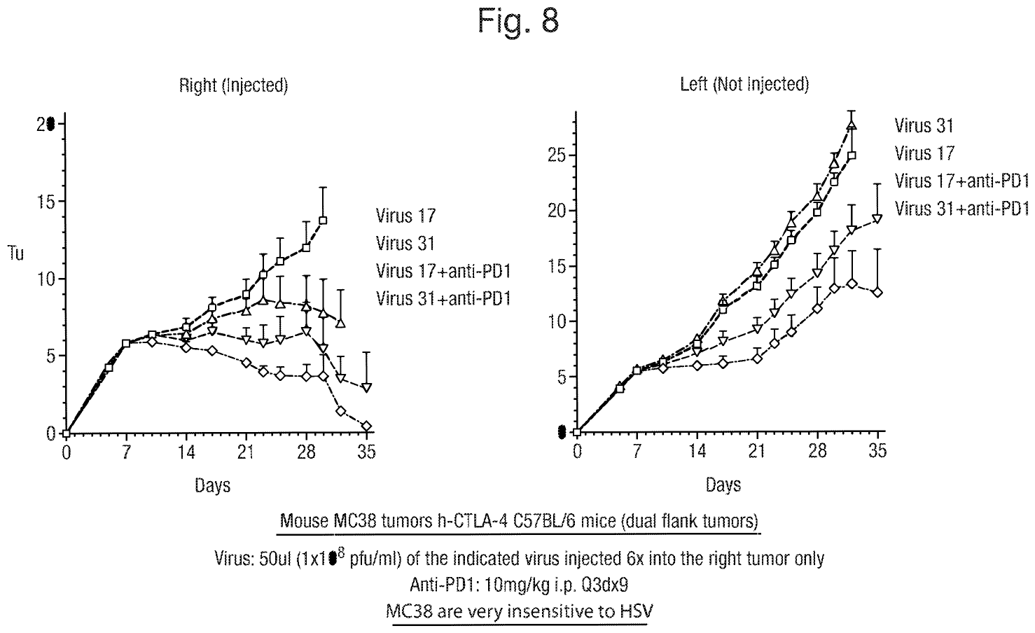

Effects on tumor growth are then observed for up to 35 days. Superior tumor control and shrinkage in injected tumors with the virus expressing anti-human CTLA-4 is seen, which is further enhanced with combined treatment with anti-PD1. Superior tumor control and shrinkage is observed in un-injected tumors when treatment with either virus is combined with anti PD1 treatment. The improvement is more marked for the virus that expresses anti CTLA4. This data is shown in FIG. 8.

### Example 5. The Effect of Combined Expression of GALV, GM-CSF and Anti-CTLA4 from an Oncolytic Virus with Anti-PD-1

A20 cells are administered into both flanks of Balb/c mice and the A20 tumors are allowed to grow to approximately 0.5 cm in diameter.

The following treatments are then administered to groups of mice (10 per group), into one flank of each mouse only 3 times per week for two weeks:
50 μl of vehicle (1 group);
Intraperitoneal anti-mouse PD1 (Bioxcell RMP-1-14 10 mg/kg every three days);
50 μl of 10⁵ pfu/ml, 10⁶ pfu/ml, or 107 pfu/ml of the HSV with only mouse GM-CSF and GALVR− inserted (3 groups);
50 μl of 10⁵ pfu/ml, 10⁶ pfu/ml, or 107 pfu/ml of the HSV with GALVR−, mouse GM-CSF and the anti-mouse CTLA-4 antibody inserted together with intraperitoneal anti-mouse PD1 (10 mg/kg every three days) (3 groups);
50 μl of 10⁵ pfu/ml, 10⁶ pfu/ml, or 107 pfu/ml of the HSV with only mouse GM-CSF and GALVR− inserted together with intraperitoneal anti-mouse PD1 (10 mg/kg every three days) (3 groups);
50 μl of 10⁵ pfu/ml, 10⁶ pfu/ml, or 107 pfu/ml of the HSV with GALVR−, mouse GM-CSF and the anti-mouse CTLA-4 antibody inserted together with intraperitoneal anti-mouse PD1 (10 mg/kg every three days) (3 groups).

Effects on tumor growth are then observed for up to one month. Superior tumor control and shrinkage in both injected and uninjected tumors with the virus expressing anti-CTLA-4 is seen, which is further enhanced with combined treatment with anti-PD1, as compared to the other groups is observed, including through an improved dose response curve.

### Example 6. The Effect of Combined Expression of GALV, GM-CSF and Anti-Human CTLA4 from an Oncolytic Virus Alone and in Combination with Anti-PD-1

MC38 cells are administered into both flanks of C57BL/6 mice engineered by gene editing to express human rather than mouse CTLA-4. This renders the mice susceptible to anti-human CTLA-4 antibodies such as ipilimumab. The MC38 tumors are allowed to grow to approximately 0.5 cm in diameter.

The following treatments are then administered to groups of mice (10 per group), into one flank of each mouse only 3 times per week for two weeks:
50 μl of vehicle (1 group);
Intraperitoneal anti-mouse PD1 (Bioxcell RMP-1-14 10 mg/kg every three days);
50 μl of 10⁵ pfu/ml, 10⁶ pfu/ml, or 107 pfu/ml of the HSV with only mouse GM-CSF and GALVR− inserted (3 groups);
50 μl of 10⁵ pfu/ml, 10⁶ pfu/ml, or 107 pfu/ml of the HSV with GALVR−, mouse GM-CSF and the anti-mouse CTLA-4 antibody inserted together with intraperitoneal anti-mouse PD1 (10 mg/kg every three days) (3 groups);
50 μl of 10⁵ pfu/ml, 10⁶ pfu/ml, or 107 pfu/ml of the HSV with only mouse GM-CSF and GALVR− inserted together with intraperitoneal anti-mouse PD1 (10 mg/kg every three days) (3 groups);
50 μl of 10⁵ pfu/ml, 10⁶ pfu/ml, or 107 pfu/ml of the HSV with GALVR−, mouse GM-CSF and the anti-mouse CTLA-4 antibody inserted together with intraperitoneal anti-mouse PD1 (10 mg/kg every three days) (3 groups).

Effects on tumor growth are then observed for up to one month. Superior tumor control and shrinkage in both injected and uninjected tumors with the virus expressing anti-CTLA-4 is seen, which is further enhanced with combined treatment with anti-PD1, as compared to the other groups is observed, including through an improved dose response curve.

### Example 7. The Effect of Combined Expression of GALV, GM-CSF, Anti-CTLA4 and an Immune Co-Stimulatory Pathway Activating Molecule from an Oncolytic Virus

The experiment in Example 3 above is repeated but mice are dosed with the viruses additionally expressing the immune co-stimulatory pathway ligands as well as expressing GALV, mGM-CSF and anti-CTLA4.

More specifically, groups of mice receive:
(1) Vehicle;
(2) Intraperitoneal anti mouse PD1;
(3) HSV with mGM-CSF, GALVR− and anti-CTLA4 inserted as in Example 2;
(4) HSV with mGM-CSF, GALVR−, anti-CTLA4 and mouse CD40L inserted;
(5) HSV with mGM-CSF, GALVR−, anti-CTLA4 and mouse 4-1 BBL inserted;
(6) HSV with mGM-CSF, GALVR−, anti-CTLA4 and mouse GITRL inserted;
(7) HSV with mGM-CSF, GALVR−, anti-CTLA4 and mouse OX40L inserted;
(8) HSV with mGM-CSF, GALVR−, anti-CTLA4 and mouse ICOSL inserted;
(9) HSV with mGM-CSF, GALVR− and anti-CTLA4 inserted as in Example 2, together with intraperitoneal anti-PD1;
(10) HSV with mGM-CSF, GALVR−, anti-CTLA4 and mouse CD40L inserted together with intraperitoneal anti-PD1;
(11) HSV with mGM-CSF, GALVR−, anti-CTLA4 and mouse 4-1 BBL inserted together with intraperitoneal anti-PD1;
(12) HSV with mGM-CSF, GALVR−, anti-CTLA4 and mouse GITRL inserted together with intraperitoneal anti-PD1;
(13) HSV with mGM-CSF, GALVR−, anti-CTLA4 and mouse OX40L inserted together with intraperitoneal anti-PD1; or
(14) HSV with mGM-CSF, GALVR−, anti-CTLA4 and mouse ICOSL inserted together with intraperitoneal anti-PD1.

Superior tumor control is seen with the viruses expressing the immune co-stimulatory ligands.

### Deposit Information

The following HSV1 strains were deposited at the ECACC, Culture Collections, Public Health England, Porton Down, Salisbury, SP4 0JG, United Kingdom on 19 Dec. 2016 by Replimune Limited and were allocated the indicated accession numbers:
RH004A—Accession Number 16121902
RH015A—Accession Number 16121903
RH018A—Accession Number 16121904
RH021A—Accession Number 16121905
RH023A—Accession Number 16121906
RH031A—Accession Number 16121907
RH040B—Accession Number 16121908
RH047A—Accession Number 16121909.

1. An oncolytic virus encoding a CTLA-4 inhibitor.

2. The virus of claim 1, wherein the CTLA-4 inhibitor is an anti-CTLA-4 antibody, or an antigen binding fragment thereof.

3. The virus of claim 2, wherein the fragment comprises a scFv molecule.

4. The virus of claim 2, wherein the fragment is a scFv molecule linked to one or more IgG1 constant regions.

5. The virus of any one of claims 2 to 4, wherein the antibody or fragment comprises a light chain variable region sequence linked to an IgG heavy chain.

6. The virus of any one of claims 2 to 5, wherein the antibody or fragment comprises (a) the light chain variable region sequence shown in SEQ ID NO: 1 and the heavy chain variable region sequence shown in SEQ ID NOs: 3; or (b) the light chain variable region sequence shown in SEQ ID NO: 11 and the heavy chain variable region sequence shown in SEQ ID NO: 12.

7. The virus of claim 6, wherein the antibody or fragment comprises (a) the amino acid sequence of SEQ ID NO: 9; or (b) the amino acid sequence of SEQ ID NO: 14.

8. The virus of claim 7, wherein the antibody or fragment is encoded by (a) the nucleotide sequence of SEQ ID NO: 10; or (b) the nucleotide sequence of SEQ ID NO: 15.

9. The virus of any one of the preceding claims, wherein the virus further comprises a GM-CSF-encoding gene.

10. The virus of any one of the preceding claims, wherein the virus further comprises an immune co-stimulatory pathway activating molecule or an immune co-stimulatory pathway activating molecule-encoding gene.

11. The virus of claim 10, wherein the immune co-stimulatory pathway activating molecule-encoding gene encodes CD40 ligand (CD40L), ICOS ligand, GITR ligand, 4-1-BB ligand, OX40 ligand, TL1A, CD30 ligand, CD27 or flt3 ligand or a modified version of any of these.

12. The virus of claim 10 or 11, wherein the immune co-stimulatory pathway activating molecule-encoding gene encodes CD40 ligand, GITR ligand, 4-1-BB ligand, OX40 ligand, ICOS ligand or a modified version of any of these.

13. The virus of any one of the preceding claims, further comprising a fusogenic protein-encoding gene.

14. The virus of claim 13 where the fusogenic protein is selected from the group consisting of vesicular stomatitis virus (VSV) G-protein, syncitin-1, syncitin-2, simian virus 5 (SV5) F-protein, measles virus (MV) H-protein, MV F-protein, respiratory syncytial virus (RSV) F-protein and a glycoprotein from gibbon ape leukemia virus (GALV), murine leukemia virus (MLV), Mason-Pfizer monkey virus (MPMV) or equine infectious anaemia virus (EIAV) from which the R peptide has been deleted.

15. The virus of claim 13 or 14, wherein the fusogenic protein is the glycoprotein from gibbon ape leukemia virus (GALV) and has the R transmembrane peptide mutated or removed (GALV-R−).

16. The virus of any one of the preceding claims, which encodes more than one immune co-stimulatory pathway activating molecule.

17. The virus of any one of the preceding claims, which is derived from a clinical isolate of a virus.

18. The virus of any one of the preceding claims, which is a modified clinical isolate of a virus, wherein the clinical isolate kills two or more tumor cell lines more rapidly and/or at a lower dose in vitro than one or more reference clinical isolates of the same species of virus.

19. The virus of any one of the preceding claims, which is selected from the group consisting of herpes viruses, pox viruses, adenoviruses, retroviruses, rhabdoviruses, paramyxoviruses and reoviruses.

20. The virus of any one of the preceding claims, which is a herpes simplex virus (HSV).

21. The virus of claim 20 which is a HSV1.

22. The virus of claim 21, wherein the HSV:
(a) does not express functional ICP34.5;
(b) does not express functional ICP47; and/or
(c) expresses the US11 gene as an immediate early gene.

23. The virus of any one of claims 20 to 22, wherein an anti-CTLA-4 inhibiting protein encoding gene has been inserted into the ICP34.5 encoding locus by insertion, partial deletion or complete deletion.

24. The virus of claim 23, wherein the anti-CTLA-4 inhibiting protein encoding gene is included in a cassette also including one or more immune stimulating gene(s) such as GM-CSF and/or an immune co-stimulatory pathway activating molecule encoding gene and/or a fusogenic protein encoding gene.

25. The virus of any one of the preceding claims, wherein the sequence encoding the CTLA-4 inhibitor, the sequence encoding GM-CSF, the sequence encoding an immune co-stimulatory pathway activating molecule and/or the sequence encoding the fusogenic protein is codon optimized so as to increase expression levels in target cells.

26. A virus according to any one of the preceding claims, which expresses three heterologous genes, wherein each of the three heterologous genes is driven by a different promoter selected from the CMV promoter, the RSV promoter, the SV40 promoter (SEQ ID) and a retroviral LTR promoter.

27. The virus of claim 26, which expresses four heterologous genes driven by each of the CMV promoter, the RSV promoter, the SV40 promoter and a retroviral LTR promoter, respectively.

28. The virus of claim 26 or 27, where the retroviral LTR is from MMLV (SEQ ID).

29. A virus according to any one of the preceding claims, which expresses three heterologous genes, wherein each of the three heterologous genes is terminated by a different poly adenylation sequence selected from the BGH, SV40, HGH and RBG poly adenylation sequences.

30. The virus of claim 29, which expresses four heterologous genes terminated by each of the BGH, SV40, HGH and RBG poly adenylation sequences, respectively.

31. The virus of any one of claims 26 to 30 which is a pox virus.

32. A pharmaceutical composition comprising a virus according to any one of claims 1 to 31 and a pharmaceutically acceptable carrier or diluent.

33. The virus of any one of claims 1 to 31 for use in a method of treating the human or animal body by therapy.

34. The virus of any one of claims 1 to 31 for use in a method of treating cancer.

35. The virus for use according to claim 34, wherein the method comprises administering a further anti-cancer agent.

36. The virus for use according to claim 35, wherein the further anti-cancer agent is selected from an agent targeting an immune co-inhibitory or immune co-stimulatory pathway, radiation and/or chemotherapy, an agent that targets a specific genetic mutation which occurs in tumors, an agent intended to induce an immune response to one or more tumor antigen(s) or neoantigen(s), a cellular product derived from T cells or NK cells, an agent intended to stimulate the STING, cGAS, TLR or other innate immune response and/or inflammatory pathway, a second virus optionally an oncolytic virus, and combinations thereof.

37. The virus for use according to claim 36, wherein the agent targeting an immune co-inhibitory pathway is a PD-1 inhibitor, a PD-L1 inhibitor, a LAG-3 inhibitor, a TIM-3 inhibitor, a VISTA inhibitor, a CSF1R inhibitor, an IDO inhibitor, a KIR inhibitor, a SLAMF7 inhibitor, a CEACAM1 inhibitor or a CD47 inhibitor, and/or the agent targeting an immune co-stimulatory pathway is a GITR agonist, a 4-1-BB agonist, an OX40 agonist, a CD40 agonist or an ICOS agonist.

38. The virus for use according to any one of claims 35 to 37, wherein the further anti-cancer agent is an antibody.

39. The virus for use according to any one of claims 35 to 38, wherein the method comprises administering an inhibitor of the indoleamine 2,3-dioxygenase (IDO) pathway and a further antagonist of an immune co-inhibitory pathway, or an agonist of an immune co-stimulatory pathway.

40. The virus for use according to any one of claims 34 to 39, wherein the virus and the further anti-cancer agent(s) are administered separately.

41. The virus for use according to any one of claims 34 to 39, wherein the virus and the further anti-cancer agent(s) are administered concurrently.

42. The virus for use according to any one of claims 34 to 41, wherein the cancer is a solid tumor.

43. A product of manufacture comprising a virus according to any one of claims 1 to 31 in a sterile vial, ampoule or syringe.

44. A method of treating cancer, which comprises administering a therapeutically effective amount of the virus of any one of claims 1 to 31 or a pharmaceutical composition according to claim 32 to a patient in need thereof.

45. A method according to claim 44, which further comprises administering a therapeutically effective amount of a further anti-cancer agent to a patient in need thereof.

46. A method according to claim 45, wherein the further anti-cancer agent is selected from the group consisting of an agent targeting an immune co-inhibitory or immune co-stimulatory pathway, radiation and/or chemotherapy, an agent that targets a specific genetic mutation which occurs in tumors, an agent intended to induce an immune response to one or more tumor antigen(s) or neoantigen(s), a cellular product derived from T cells or NK cells, an agent intended to stimulate the STING, cGAS, TLR or other innate immune response and/or inflammatory pathway, a second virus optionally an oncolytic virus, and combinations thereof.

47. A method according to claim 46, wherein the agent targeting an immune co-inhibitory pathway is a PD-1 inhibitor, a PD-L1 inhibitor, a LAG-3 inhibitor, a TIM-3 inhibitor, a VISTA inhibitor, a CSF1R inhibitor, an IDO inhibitor, a KIR inhibitor, a SLAMF7 inhibitor, a CEACAM1 inhibitor or a CD47 inhibitor, and/or the agent targeting an immune co-stimulatory pathway is a GITR agonist, a 4-1-BB agonist, an OX40 agonist, a CD40 agonist or an ICOS agonist.

48. A method according to any one of claims 45 to 47, wherein the further anti-cancer agent comprises an antibody.

49. A method according to any one of claims 45 to 48, wherein the virus and the further anti-cancer agent(s) are administered separately.

50. A method according to any one of claims 45 to 48, wherein the virus and the further anti-cancer agent(s) are administered concurrently.

51. A method according to any one of claims 44 to 50, wherein the cancer is a solid tumor.

52. Use of the virus of any one of claims 1 to 31 in the manufacture of a medicament for use in a method of treating cancer.

53. Use according to claim 52, wherein the method comprises administering a further anti-cancer agent.


SEQUENCE LISTING (46 sequences)

SEQ ID NO: 1 - protein, 108 aa, Homo sapiens
IVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKR

SEQ ID NO: 2 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 4 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 21 aa, Mus musculus
METDTLLLWVLLLWVPGSTGD

SEQ ID NO: 8 - protein, 15 aa, synthetic construct
  REGION 1..15: note = (Gly4Ser)3 linker
GGGGSGGGGSGGGGS

SEQ ID NO: 9 - protein, 489 aa, Homo sapiens
METDTLLLWVLLLWVPGSTGDIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1470 bp, Homo sapiens
  source 1..1470: mol_type = other DNA
atggaaactgacaccctgctgctgtgggtcctgctgctgtgggtgcctggatccaccggcgatatcgtgctgacccagtctcctggcacactgagtctgtcaccaggggagcgagcaacactgtcttgtagagccagccagtctgtgggaagctcctacctggcttggtatcagcagaagccaggccaggcacccaggctgctgatctacggagccttcagccgggccactggcattccagacaggttctctggaagtggctcagggaccgacttcaccctgaccatcagccgactggagcccgaagacttcgccgtgtactattgccagcagtacggctctagtccttggacttttggacagggcaccaaagtggagatcaagcgcggcgggggaggctctgggggaggcgggagtggaggcgggggatcacaggtccagctggtggaaagcggcgggggagtggtccagccaggccggagcctgcggctgagctgcgccgcttcaggattcacattttcaagctataccatgcactgggtccggcaggcaccagggaagggactggagtgggtgaccttcatcagctatgacggcaacaacaagtattacgctgattccgtgaaagggaggtttaccattagccgcgacaactccaaaaatacactgtacctgcagatgaacagcctgcgggccgaggatactgctatctactattgcgcaagaaccgggtggctgggacccttcgactattggggccaggggactctggtcaccgtgtcctctgataagacacacacatgccctccctgtcctgcaccagagctgctgggcgggccatccgtgttcctgtttccacccaagcctaaagacaccctgatgatcagccggacacctgaagtcacttgcgtggtcgtggacgtgagtcacgaggatccagaagtcaagtttaactggtacgtggatggcgtcgaggtgcataatgccaagaccaaacctcgcgaggaacagtacaatagcacatatcgagtcgtgtccgtcctgactgtgctgcatcaggattggctgaacggcaaagagtataagtgcaaagtgagcaataaggcactgcctgccccaatcgagaaaacaatttccaaggctaaaggccagcccagggaacctcaggtgtacactctgcctccaagtcgcgaggaaatgaccaagaaccaggtgagcctgacctgtctggtgaaagggttctatccatcagacattgcagtggagtgggaaagcaatggacagcccgaaaacaattacaagaccacaccccctgtgctggacagcgatggctccttctttctgtattctaagctgactgtggataaaagtcgctggcagcaggggaacgtctttagctgttccgtgatgcatgaggctctgcacaatcattacacacagaagtctctgagtctgtcacccggcaaatga

SEQ ID NO: 11 - protein, 127 aa, Mus musculus
IRRADIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSGS

SEQ ID NO: 12 - protein, 134 aa, Mus musculus
EAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAPRSSR

SEQ ID NO: 13 - protein, 356 aa, Mus musculus
EAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAPRSSRGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 14 - protein, 519 aa, Mus musculus
METDTLLLWVLLLWVPGSTGDIRRADIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSGSGGGGSGGGGSGGGGSEAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAPRSSRGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - DNA, 1560 bp, Mus musculus
  source 1..1560: mol_type = other DNA
atggaaactgatactctgctgctctgggtgctgctcctctgggtgcctggttcaactggggacattcgacgggctgacattgtgatgacccagaccacactgagcctgcccgtgtccctgggcgaccaggccagcatctcctgccggagctcccagtctatcgtgcacagcaacggaaacacatacctggagtggtatctgcagaagcctggccagtccccaaagctgctgatctacaaggtgtccaacaggttcagcggcgtgcctgaccgcttttctggaagcggctccggaacagatttcaccctgaagatcagcagggtggaggctgaggacctgggcgtgtactactgcttccagggatcccacgtgccttacacctttggcggaggcacaaagctggagatcaagagagccgatgctgctccaaccgtgtctggaagcggaggcgggggttctggaggcggtgggagcggtggcggagggtctgaggctaagctgcaggagagcggccccgtgctggtgaagcctggagccagcgtgaagatgtcctgtaaggcttctggatacaccttcacagactactacatgaactgggtgaagcagagccacggcaagtccctggagtggatcggagtgatcaacccttacaacggcgacacctcttacaaccagaagtttaagggcaaggccaccctgacagtggataagtctagctccaccgcttacatggagctgaacagcctgacatccgaggattctgccgtgtactactgtgctaggtactacggaagctggttcgcctactggggccagggaacactgatcaccgtgtccacagccaagaccacaccccctagcgtgtaccccctggctcctaggtctagcagaggctgcaagccatgcatctgtaccgtgcccgaggtgagcagcgtgttcatctttccacccaagcccaaggacgtgctgaccatcacactgacccctaaggtgacatgcgtggtggtggatatcagcaaggacgatccagaggtgcagttctcctggtttgtggacgatgtggaggtgcacaccgcccagacacagccaagggaggagcagttcaactccacctttagatccgtgtctgagctgcccatcatgcaccaggactggctgaacggaaaggagttcaagtgccgggtgaactccgccgcttttcctgctccaatcgagaagaccatctctaagacaaagggccgcccaaaggctccacaggtgtacaccatccctccacccaaggagcagatggctaaggataaggtgagcctgacctgtatgatcacagacttctttcccgaggatatcacagtggagtggcagtggaacggacagcctgccgagaactacaagaacacccagccaatcatggacacagatggctcttacttcgtgtacagcaagctgaacgtgcagaagtctaactgggaggctggcaacaccttcacctgcagcgtgctgcacgaaggtctccataatcaccacaccgaaaagagcctcagtcacagccctgggaaatga

SEQ ID NO: 16 - DNA, 1574 bp, Mus musculus
  source 1..1574: mol_type = other DNA
cttaagatggaaactgatactctgctgctctgggtgctgctcctctgggtgcctggttcaactggggacattcgacgggctgacattgtgatgacccagaccacactgagcctgcccgtgtccctgggcgaccaggccagcatctcctgccggagctcccagtctatcgtgcacagcaacggaaacacatacctggagtggtatctgcagaagcctggccagtccccaaagctgctgatctacaaggtgtccaacaggttcagcggcgtgcctgaccgcttttctggaagcggctccggaacagatttcaccctgaagatcagcagggtggaggctgaggacctgggcgtgtactactgcttccagggatcccacgtgccttacacctttggcggaggcacaaagctggagatcaagagagccgatgctgctccaaccgtgtctggaagcggaggcgggggttctggaggcggtgggagcggtggcggagggtctgaggctaagctgcaggagagcggccccgtgctggtgaagcctggagccagcgtgaagatgtcctgtaaggcttctggatacaccttcacagactactacatgaactgggtgaagcagagccacggcaagtccctggagtggatcggagtgatcaacccttacaacggcgacacctcttacaaccagaagtttaagggcaaggccaccctgacagtggataagtctagctccaccgcttacatggagctgaacagcctgacatccgaggattctgccgtgtactactgtgctaggtactacggaagctggttcgcctactggggccagggaacactgatcaccgtgtccacagccaagaccacaccccctagcgtgtaccccctggctcctaggtctagcagaggctgcaagccatgcatctgtaccgtgcccgaggtgagcagcgtgttcatctttccacccaagcccaaggacgtgctgaccatcacactgacccctaaggtgacatgcgtggtggtggatatcagcaaggacgatccagaggtgcagttctcctggtttgtggacgatgtggaggtgcacaccgcccagacacagccaagggaggagcagttcaactccacctttagatccgtgtctgagctgcccatcatgcaccaggactggctgaacggaaaggagttcaagtgccgggtgaactccgccgcttttcctgctccaatcgagaagaccatctctaagacaaagggccgcccaaaggctccacaggtgtacaccatccctccacccaaggagcagatggctaaggataaggtgagcctgacctgtatgatcacagacttctttcccgaggatatcacagtggagtggcagtggaacggacagcctgccgagaactacaagaacacccagccaatcatggacacagatggctcttacttcgtgtacagcaagctgaacgtgcagaagtctaactgggaggctggcaacaccttcacctgcagcgtgctgcacgaaggtctccataatcaccacaccgaaaagagcctcagtcacagccctgggaaatgaggcgcgcc

SEQ ID NO: 17 - DNA, 1484 bp, Homo sapiens
  source 1..1484: mol_type = other DNA
cttaagatggaaactgacaccctgctgctgtgggtcctgctgctgtgggtgcctggatccaccggcgatatcgtgctgacccagtctcctggcacactgagtctgtcaccaggggagcgagcaacactgtcttgtagagccagccagtctgtgggaagctcctacctggcttggtatcagcagaagccaggccaggcacccaggctgctgatctacggagccttcagccgggccactggcattccagacaggttctctggaagtggctcagggaccgacttcaccctgaccatcagccgactggagcccgaagacttcgccgtgtactattgccagcagtacggctctagtccttggacttttggacagggcaccaaagtggagatcaagcgcggcgggggaggctctgggggaggcgggagtggaggcgggggatcacaggtccagctggtggaaagcggcgggggagtggtccagccaggccggagcctgcggctgagctgcgccgcttcaggattcacattttcaagctataccatgcactgggtccggcaggcaccagggaagggactggagtgggtgaccttcatcagctatgacggcaacaacaagtattacgctgattccgtgaaagggaggtttaccattagccgcgacaactccaaaaatacactgtacctgcagatgaacagcctgcgggccgaggatactgctatctactattgcgcaagaaccgggtggctgggacccttcgactattggggccaggggactctggtcaccgtgtcctctgataagacacacacatgccctccctgtcctgcaccagagctgctgggcgggccatccgtgttcctgtttccacccaagcctaaagacaccctgatgatcagccggacacctgaagtcacttgcgtggtcgtggacgtgagtcacgaggatccagaagtcaagtttaactggtacgtggatggcgtcgaggtgcataatgccaagaccaaacctcgcgaggaacagtacaatagcacatatcgagtcgtgtccgtcctgactgtgctgcatcaggattggctgaacggcaaagagtataagtgcaaagtgagcaataaggcactgcctgccccaatcgagaaaacaatttccaaggctaaaggccagcccagggaacctcaggtgtacactctgcctccaagtcgcgaggaaatgaccaagaaccaggtgagcctgacctgtctggtgaaagggttctatccatcagacattgcagtggagtgggaaagcaatggacagcccgaaaacaattacaagaccacaccccctgtgctggacagcgatggctccttctttctgtattctaagctgactgtggataaaagtcgctggcagcaggggaacgtctttagctgttccgtgatgcatgaggctctgcacaatcattacacacagaagtctctgagtctgtcacccggcaaatgaggcgcgcc

SEQ ID NO: 18 - DNA, 426 bp, Mus musculus
  source 1..426: mol_type = other DNA
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatga

SEQ ID NO: 19 - DNA, 426 bp, Mus musculus
  source 1..426: mol_type = other DNA
atgtggctccagaacctcctcttcctcggtatcgtcgtgtattcactctccgcacctactcgctcacctatcactgtcaccagaccctggaagcacgtggaggccatcaaggaggctctgaacctgctggacgatatgccagtgaccctgaacgaggaggtggaggtggtgagcaacgagttctcctttaagaagctgacctgcgtgcagacaaggctgaagatcttcgagcagggcctgagaggaaactttaccaagctgaagggcgccctgaacatgaccgcttcttactaccagacatactgcccccctacccccgagacagactgtgagacacaggtgaccacatacgccgacttcattgatagcctgaaaacattcctgaccgacattccatttgagtgtaagaagcccgtccagaagtaa

SEQ ID NO: 20 - DNA, 435 bp, Homo sapiens
  source 1..435: mol_type = other DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 21 - DNA, 435 bp, Homo sapiens
  source 1..435: mol_type = other DNA
atgtggctgcagtccctgctgctgctgggcaccgtcgcctgttctatttccgcacccgcaaggtcaccaagtccatctactcagccttgggagcacgtgaacgcaatccaggaggcacggcggctgctgaacctgagccgggacaccgccgccgagatgaacgagacagtggaagtgatcagcgagatgttcgatctgcaggagcccacctgcctgcagacaaggctggagctgtacaagcagggcctgcgcggctctctgaccaagctgaagggcccactgacaatgatggccagccactataagcagcactgcccccctacccccgagacaagctgtgccacccagatcatcacattcgagtcctttaaggagaacctgaaggattttctgctggtcattccatttgattgttgggagcccgtccaggagtaa

SEQ ID NO: 22 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 23 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 24 - DNA, 2010 bp, Gibbon ape leukemia virus
  source 1..2010: mol_type = other DNA
atggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcagttaaaatttaa

SEQ ID NO: 25 - DNA, 2013 bp, Gibbon ape leukemia virus
  source 1..2013: mol_type = other DNA
accatggtcctgctgcctgggtctatgctgctgacttctaacctgcaccacctgcgacaccagatgtctcccggctcatggaaacggctgatcatcctgctgagctgcgtgttcggaggaggaggcacctccctgcagaacaagaatcctcaccagccaatgaccctgacatggcaggtgctgtcccagacaggcgacgtggtgtgggataccaaggcagtgcagccaccttggacatggtggcccaccctgaagcctgacgtgtgcgccctggccgcctccctggagtcttgggacatccccggcacagacgtgagcagcagcaagagggtgagaccacccgactctgattatacagccgcctacaagcagatcacctggggcgccatcggctgtagctatcctcgggcccgcacaaggatggccagctccaccttttacgtgtgcccacgcgacggaaggaccctgtctgaggcaaggagatgtggcggcctggagagcctgtattgcaaggagtgggattgtgagaccacaggcacaggctactggctgtctaagtctagcaaggacctgatcaccgtgaagtgggatcagaacagcgagtggacacagaagttccagcagtgccaccagaccggctggtgtaatcccctgaagatcgactttacagataagggcaagctgtccaaggactggatcaccggcaagacatggggcctgagattctacgtgtctggccaccctggcgtgcagtttacaatccggctgaagatcaccaacatgccagcagtggcagtgggaccagacctggtgctggtggagcagggacctccacgcacctccctggccctgccccctccactgccccctagggaggccccaccccctagcctgcccgattctaacagcacagccctggccacctccgcccagacccctacagtgcgcaagaccatcgtgacactgaataccccaccccctaccacaggcgacaggctgttcgatctggtgcagggcgcctttctgacactgaacgccaccaatcctggcgcaaccgagagctgctggctgtgcctggctatgggcccaccctactatgaggcaatcgcctcctctggagaggtggcatattccacagacctggatagatgcagatggggcacccagggcaagctgaccctgacagaggtgtctggccacggcctgtgcatcggcaaggtgccattcacacaccagcacctgtgcaaccagaccctgagcatcaatagctccggcgaccaccagtacctgctgccaagcaaccactcctggtgggcatgctccacaggactgaccccatgtctgagcaccagcgtgttcaaccagaccagagacttttgtatccaggtgcagctgatccctcggatctactattacccagaggaggtgctgctgcaggcctatgataattcccacccaagaacaaagagggaggccgtgtctctgaccctggccgtgctgctgggactgggaatcacagcaggaatcggcacaggcagcaccgccctgatcaagggaccaatcgacctgcagcagggactgacctccctgcagatcgccatcgacgccgatctgagagccctgcaggacagcgtgtccaagctggaggattctctgacctctctgagcgaggtggtgctgcagaacaggaggggcctggacctgctgttcctgaaggagggaggactgtgcgccgccctgaaggaggagtgctgtttttatatcgaccactctggcgccgtgcgggatagcatgaagaagctgaaggagaagctggataagcgccagctggagaggcagaagagccagaattggtacgagggctggttcaacaattccccctggtttaccacactgctgtctaccatcgcaggacctctgttattactgctgctgctgctgatcctgggcccatgtatcatcaacaagctggtgcagtttatcaacgaccgaatctccgcagtgaaaatctaa

SEQ ID NO: 26 - protein, 669 aa, Gibbon ape leukemia virus
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKI

SEQ ID NO: 27 - DNA, 759 bp, synthetic construct
  misc_feature 1..759: note = Human-Mouse hybrid
  source 1..759: mol_type = other DNA
atgatcgagacctacaatcagacaagcccacggtccgccgcaaccggactgcctatcagcatgaagatcttcatgtacctgctgaccgtgtttctgatcacacagatgatcggctccgccctgttcgccgtgtatctgcacaggagactggacaagatcgaggatgagcgcaatctgcacgaggacttcgtgtttatgaagaccatccagcggtgcaacacaggcgagaggagcctgtctctgctgaattgtgaggagatcaagtcccagttcgagggctttgtgaaggatatcatgctgaacaaggaggagacaaagaaggacgaggatccacagatcgcagcacacgtggtgtccgaggcaaactctaatgccgccagcgtgctgcagtgggccaagaagggctactataccatgaagtctaacctggtgacactggagaatggcaagcagctgaccgtgaagaggcagggcctgtactatatctatgcccaggtgacattctgctctaacagagaggcaagctcccaggcacccttcatcgtgggactgtggctgaagccctctagcggcagcgagaggatcctgctgaaggccgccaatacccactcctctagccagctgtgcgagcagcagtccatccacctgggaggcgtgttcgagctgcagcctggagccagcgtgttcgtgaacgtgacagacccatctcaggtgagccacggcaccggcttcacaagctttggcctgctgaagctgtga

SEQ ID NO: 28 - protein, 252 aa, synthetic construct
  REGION 1..252: note = Human-Mouse hybrid
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSIHLGGVFELQPGASVFVNVTDPSQVSHGTGFTSFGLLKL

SEQ ID NO: 29 - DNA, 1416 bp, Homo sapiens
  source 1..1416: mol_type = other DNA
atgctgccctttctgagcatgctggtgctgctggtgcagcctctgggaaacctgggagccgagatgaagagcctgtcccagagatctgtgcctaacacctgcacactggtcatgtgcagccccaccgagaatggactgcctggaagggacggaagggatggaagggagggccctcggggcgagaagggcgacccaggactgcctggaccaatgggactgagcggactgcagggaccaacaggacctgtgggaccaaagggagagaacggatccgccggagagccaggccctaagggcgagaggggcctgtctggcccccctggcctgccaggcatcccaggccccgccggcaaggagggcccatccggcaagcagggcaatatcggcccccagggcaagcctggcccaaagggcgaggcaggaccaaagggagaagtgggagcacctggcatgcagggatccaccggagcaaagggatctacaggaccaaagggcgagcgcggcgccccaggcgtgcagggcgcccccggcaatgcaggagcagcaggaccagcaggacctgcaggcccacagggcgcccctggctctaggggcccacccggcctgaagggcgacaggggagtgcctggcgataggggcatcaagggagagagcggactgccagattccgccgccctgaggcagcagatggaggccctgaagggcaagctgcagaggctggaggtggccttctcccactaccagaaggccgccctgtttccagacggccacaggagactggacaagatcgaggatgagcgcaacctgcacgaggatttcgtgtttatgaagaccatccagagatgcaacacaggcgagcggtctctgagcctgctgaattgtgaggagatcaagtctcagttcgagggctttgtgaaggacatcatgctgaacaaggaggagaccaagaaggagaatagcttcgagatgcagaagggcgatcagaatccccagatcgcagcacacgtgatcagcgaggcaagctccaagaccacatccgtgctgcagtgggccgagaagggctactataccatgtccaacaatctggtgacactggagaacggcaagcagctgaccgtgaagagacagggcctgtactatatctatgcccaggtgacattctgctctaatcgggaggcctctagccaggccccttttatcgcctctctgtgcctgaagagcccaggcagattcgagcggatcctgctgagggccgccaacacccactcctctgccaagccatgcggacagcagagcatccacctgggaggcgtgttcgagctgcagccaggagcctccgtgtttgtgaatgtgacagacccatcccaggtgtctcacggaaccggcttcacatcctttggcctgctgaagctgtga

SEQ ID NO: 30 - protein, 471 aa, Homo sapiens
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 31 - DNA, 1412 bp, Mus musculus
  source 1..1412: mol_type = other DNA
atgctgcccttcctgagcatgctggtgctgctggtgcagcctctgggcaatctgggcgccgagatgaagtccctgtctcagaggagcgtgccaaacacctgcacactggtcatgtgctctccaaccgagaatggactgccaggaagggacggaagagatggaagggagggaccaaggggagagaagggcgaccctggactgcctggaccaatgggactgtccggactgcagggaccaacaggccctgtgggaccaaagggagagaatggaagcgccggagagccaggacctaagggagagaggggcctgtccggcccccctggcctgcctggcatcccaggccccgccggcaaggagggcccttctggcaagcagggcaacatcggaccacagggcaagcctggaccaaagggagaggcaggaccaaagggagaagtgggagcacccggcatgcagggcagcaccggagcaaagggatccaccggccctaagggagagagaggagcacctggagtgcagggcgccccaggcaatgcaggagcagcaggaccagcaggacctgcaggcccacagggcgccccaggcagccggggcccacccggcctgaagggcgacaggggagtgccaggcgataggggcatcaagggagagtccggactgccagactctgccgccctgaggcagcagatggaggccctgaagggcaagctgcagaggctggaggtggccttctcccactaccagaaggccgccctgtttccagacggacacaggagactggataaggtggaggaggaggtgaacctgcacgaggatttcgtgttcatcaagaagctgaagaggtgcaacaagggcgagggcagcctgtccctgctgaattgtgaggagatgcggcgccagttcgaggacctggtgaaggatatcaccctgaacaaggaggagaagaaggagaattcttttgagatgcagaggggcgacgaggatcctcagatcgcagcacacgtggtgtccgaggcaaactctaatgccgccagcgtgctgcagtgggccaagaagggctactataccatgaagtctaacctggtcatgctggagaatggcaagcagctgacagtgaagagagagggcctgtactacgtgtacacccaggtgacattctgcagcaacagagagcccagctcccagcggccttttatcgtgggcctgtggctgaagccctctatcggaagcgagaggatcctgctgaaggcagccaatacccactctagctcccagctgtgcgagcagcagtccgtgcacctgggaggcgtgttcgagctgcaggcaggagcaagcgtgttcgtgaacggacagaggccagccaggtcatccacagagtgggcttctctagctttggcctgctgaagctgtga

SEQ ID NO: 32 - protein, 470 aa, Mus musculus
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSIGSERILLKAANTHSSSQLCEQQSVHLGGVFELQAGASVFVNVTEASQVIHRVGFSSFGLLKL

SEQ ID NO: 33 - DNA, 786 bp, Homo sapiens
  source 1..786: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 34 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 35 - DNA, 783 bp, Mus musculus
  source 1..783: mol_type = other DNA
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga

SEQ ID NO: 36 - protein, 260 aa, Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 37 - DNA, 632 bp, synthetic construct
  misc_feature 1..632: note = CMV promoter
  source 1..632: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 38 - DNA, 394 bp, synthetic construct
  misc_feature 1..394: note = RSV promoter
  source 1..394: mol_type = other DNA
tgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctcc

SEQ ID NO: 39 - DNA, 188 bp, synthetic construct
  misc_feature 1..188: note = BGH polyA
  source 1..188: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagac

SEQ ID NO: 40 - DNA, 249 bp, synthetic construct
  misc_feature 1..249: note = SV40 late polyA
  source 1..249: mol_type = other DNA
gacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 41 - DNA, 99 bp, synthetic construct
  misc_feature 1..99: note = Rabbit beta-globulin polyA
  source 1..99: mol_type = other DNA
gacctctggctaataaaggaaatttattttcattgcaatagtgtgttggaattttttgtgtctctcactcggaaggacatatgggagggcaaatcattt

SEQ ID NO: 42 - DNA, 723 bp, synthetic construct
  misc_feature 1..723: note = GFP
  source 1..723: mol_type = other DNA
accatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 43 - DNA, 454 bp, synthetic construct
  misc_feature 1..454: note = MoMuLV LTR
  source 1..454: mol_type = other DNA
ttaattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaag

SEQ ID NO: 44 - DNA, 1349 bp, synthetic construct
  misc_feature 1..1349: note = EF1alpha promoter
  source 1..1349: mol_type = other DNA
ttaattaagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcggttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttaag

SEQ ID NO: 45 - DNA, 345 bp, synthetic construct
  misc_feature 1..345: note = SV40 enhancer promoter
  source 1..345: mol_type = other DNA
gctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagct

SEQ ID NO: 46 - DNA, 481 bp, synthetic construct
  misc_feature 1..481: note = HGH polyA
  source 1..481: mol_type = other DNA
gacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttt